(19) United States
(12) Patent Application Publication
(10) Pub. No.: US 2026/0069419 A1
(43) Pub. Date: Mar. 12, 2026

(54) PRESSURE MONITORING FOR DEVICE MISUSE

(71) Applicant: Boston Scientific Scimed, Inc., Maple Grove, MN (US)

(72) Inventors: Brian Thomas Swanson, Fridley, MN (US); Kevin James Ivanca, Minneapolis, MN (US); Keith R. Maile, New Brighton, MN (US); Matthew Philip Dion, Saint Francis, MN (US); Daniel Lindquist, Shoreview, MN (US)

(21) Appl. No.: 19/317,837

(22) Filed: Sep. 3, 2025

Related U.S. Application Data
(60) Provisional application No. 63/692,415, filed on Sep. 9, 2024.

(51) Int. Cl. A61F 2/26; A61F 2/48; G16H 40/67

(52) U.S. Cl. CPC A61F 2/26; A61F 2/482; A61F 2/484; G16H 40/67; A61F 2250/0013

(57) ABSTRACT

An implantable device includes a battery, a fluid reservoir, an inflatable member, a piezoelectric pump, driver circuitry, and a processor. The battery is configured to store electrical energy. The inflatable member is configured to receive fluid to place the inflatable member in an inflated configuration. The piezoelectric pump is configured to pump fluid from the inflatable member to the fluid reservoir. The driver circuitry is configured to provide a waveform of electrical energy to drive the piezoelectric pump to pump fluid from the inflatable member to the fluid reservoir. The processor is configured to, based on a fluid pressure detected within the inflatable member, cause the driver circuitry to provide a waveform of electrical energy from the battery to the piezoelectric pump to cause the piezoelectric pump to pump fluid from the inflatable member to the fluid reservoir.

### CROSS-REFERENCE TO RELATED APPLICATION

This application claims priority to U.S. Provisional Patent Application No. 63/692,415, filed on Sep. 9, 2024, entitled “PRESSURE MONITORING FOR DEVICE MISUSE”, the disclosure of which is incorporated by reference herein in its entirety.

### TECHNICAL FIELD

This disclosure relates generally to bodily implants, and more specifically to bodily implants including a fluid control system having one or more piezoelectric-operated pumps and/or valves.

### BACKGROUND

Active implantable fluid-operated inflatable devices can include one or more pumps that regulate the flow of fluid between different portions of the implantable device. One or more valves can be positioned within fluid passageways of the device to direct and control the flow of fluid to achieve inflation, deflation, pressurization, depressurization, activation, deactivation and the like of different fluid-filled components of the device. In some implantable fluid-operated devices, an implantable pumping device may be manually operated by the user to provide for the transfer of fluid between a reservoir and the fluid-filled implant components of the device. In some situations, manual operation of the pumping device may make it difficult to achieve consistent inflation, deflation, pressurization, depressurization, activation, deactivation and the like of the fluid-filled implant components. Inconsistent inflation, deflation, pressurization, depressurization, activation and/or deactivation of the fluid-filled implant device(s) may adversely affect patient comfort, efficacy of the device, and the overall patient experience. Some implantable fluid-operated devices include an electronic control system including an electronically controlled manifold providing for the transfer of fluid within the implantable fluid-operated device.

The use of the electronic control system may provide for more accurate actuation and control of the flow of fluid between components of the inflatable device, thus improving performance and efficacy of the device, as well as patient comfort and safety. The electronic control system may include one or more electronically-operated pumps and one or more valves to control the flow of fluid in the system, and the pumps and valves may be operated by way of piezoelectric elements associated with the pumps and valves. Electronically-operated pumps and valves are complex systems and may become damaged and/or cause patient injury if they are misused by the patient.

Thus, a need exists to monitor the components of implantable devices having electronically-operated pumps and valves and to take corrective action in the event of patient misuse or other unintended or out of the ordinary occurrences.

### SUMMARY

According to a general aspect, an implantable includes a battery, a fluid reservoir, an inflatable member, a piezoelectric pump, driver circuitry, and a processor. The battery is configured to store electrical energy. The inflatable member is configured to receive fluid to place the inflatable member in an inflated configuration. The piezoelectric pump is fluidically connected between the fluid reservoir and the inflatable member and configured to pump fluid from the inflatable member to the fluid reservoir. The driver circuitry is configured to receive electrical energy from the battery and to provide a waveform of electrical energy to drive the piezoelectric pump to pump fluid from the inflatable member to the fluid reservoir. The processor is configured to, based on a fluid pressure detected within the inflatable member, cause the driver circuitry to provide a waveform of electrical energy from the battery to the piezoelectric pump to cause the piezoelectric pump to pump fluid from the inflatable member to the fluid reservoir.

In some implementations, the implantable device includes a pressure sensor configured to measure a fluid pressure in a fluidic circuit that includes the fluid reservoir, the piezoelectric pump, and the inflatable member. In some implementations, t he implantable device of claim includes a pressure sensor connected to the fluidic circuit between the piezoelectric pump and the inflatable member to measure a fluid pressure in the inflatable member.

In some implementations, the processor is further configured to determine when the fluid pressure detected within the inflatable member has exceeded a threshold value. In some implementations, the processor is further configured to determine when a voltage spike occurs on the piezoelectric pump.

In some implementations, the implantable device includes a piezoelectric valve fluidically connected between the fluid reservoir and the inflatable member. In some implementations, the implantable device includes a piezoelectric valve fluidically connected between the fluid reservoir and the inflatable member, wherein the processor is further configured to determine when a voltage spike occurs on the piezoelectric valve.

According to a general aspect, an implantable device includes a battery, a fluid reservoir, a piezoelectric valve, driver circuitry, and a processor. The battery is configured to store electrical energy. The inflatable member is configured to receive fluid to place the inflatable member in an inflated configuration. The piezoelectric valve is fluidically connected between the fluid reservoir and the inflatable member. The driver circuitry is configured to receive electrical energy from the battery and to provide a waveform of electrical energy to the piezoelectric valve. The processor is configured to, based on a fluid pressure detected within the inflatable member, cause the driver circuitry to provide a waveform of electrical energy from the battery to the piezoelectric valve to cause the piezoelectric valve to allow fluid to pass from the inflatable member to the fluid reservoir.

In some implementations, the implantable device includes a pressure sensor configured to measure a fluid pressure in a fluidic circuit that includes the fluid reservoir, the piezoelectric pump, and the inflatable member. In some implementations, t he implantable device includes a pressure sensor connected to the fluidic circuit between the piezoelectric pump and the inflatable member to measure a fluid pressure in the inflatable member.

In some implementations, the processor is configured to determine when the fluid pressure detected within the inflatable member has exceeded a threshold value. In some implementations, the processor is configured to determine when a voltage spike occurs on the piezoelectric valve.

In some implementations, the implantable device includes a piezoelectric pump fluidically connected between the fluid reservoir and the inflatable member. In some implementations, the implantable device includes a piezoelectric pump fluidically connected between the fluid reservoir and the inflatable member, wherein the processor is further configured to determine when a voltage spike occurs on the piezoelectric pump.

In some implementations, the implantable device includes a first pressure sensor configured to measure a fluid pressure in a fluidic circuit that includes the fluid reservoir, the piezoelectric pump, and the inflatable member; and a second pressure sensor configured to measure a fluid pressure in a fluidic circuit that includes the fluid reservoir, the piezoelectric pump, and the inflatable member.

According to another general aspect, a method of operating an implantable device that includes a battery, a fluid reservoir, an inflatable member, a piezoelectric pump fluidically connected between the fluid reservoir and the inflatable member, the method includes providing fluid to the inflatable member to place the inflatable member in an inflated configuration; monitoring a fluid pressure in a fluidic circuit that includes the fluid reservoir, the piezoelectric pump, and the inflatable member after the inflatable member has been placed in the inflated configuration; and based on a fluid pressure measured, causing fluid to move from the inflatable member to the fluid reservoir.

In some implementations, the implantable device includes a pressure sensor configured to measure a fluid pressure within the fluidic circuit, the monitoring includes monitoring the fluid pressure in the fluidic circuit via the pressure sensor. In some implementations, the monitoring the fluid pressure in the fluidic circuit includes detecting a voltage spike on the piezoelectric pump.

In some implementations, the implantable device includes a piezoelectric valve, the monitoring the fluid pressure in the fluidic circuit includes detecting a voltage spike on the piezoelectric valve.

In some implementations, the causing the fluid to move from the inflatable member to the fluid reservoir includes causing the piezoelectric pump to move the fluid from the inflatable member to the fluid reservoir.

### BRIEF DESCRIPTION OF THE DRAWINGS

FIG. 1 is a block diagram of an implantable fluid-operated inflatable device.

FIG. 2A illustrates a system including an example implantable fluid-operated inflatable device.

FIG. 2B illustrates a system including an example implantable fluid-operated inflatable device.

FIG. 3 is a schematic diagram of a fluidic architecture of an implantable fluid-operated inflatable device.

FIG. 4A is an exploded view of an example valve device of a fluid control system of a fluid-operated inflatable device.

FIG. 4B is another exploded view of the example valve device shown in FIG. 4A.

FIG. 4C is a cross-sectional view of the example valve device shown in FIG. 4A, in a closed position.

FIG. 4D is a cross-sectional view of the example valve device shown in FIG. 4A, in an open position.

FIG. 5A is a schematic view of an example valve device including an example auxiliary flow control device, with the example valve device in an open position.

FIG. 5B is a schematic view of an example valve device including an example auxiliary flow control device, with the example valve device in a closed position.

FIG. 6A is an exploded view of an example pump device of a fluid control system of a fluid-operated inflatable device.

FIG. 6B is a cross-sectional view of the example pump device shown in FIG. 6A, in an open position.

FIGS. 7A, 7B, and 7C are cross-sectional views of example pump devices that includes a filter for capturing particulate matter in the fluid flow and/or for blocking the particulate matter from entering certain parts of the fluidic system (e.g., for blocking particulate matter from entering a pump chamber of the device).

FIG. 8 is a schematic end view of a filter foil.

FIGS. 9A, 9B, and 9C are cross-sectional views of example pump devices that includes a filter for capturing particulate matter in the fluid flow and/or for blocking the particulate matter from entering certain parts of the fluidic system (e.g., for blocking particulate matter from entering a pump chamber of the device).

FIG. 10 is a cross-sectional view of the valve device of FIGS. 5A and 5B, but also including a filter located at an end of a second fluid passageway and a filter located within a first fluid passageway.

FIG. 11A is a graph of the voltage amplitude of an example waveform that can be a provided by the driver to the piezoelectric element to drive the piezoelectric element to cause a pump associated with the piezoelectric element to pump fluid.

FIG. 11B is a graph of the voltage amplitude of another example waveform that can be a provided by the driver to the piezoelectric element to drive the piezoelectric element to cause a pump associated with the piezoelectric element to pump fluid.

FIG. 11C is a graph of the voltage amplitude of another example waveform that can be a provided by the driver to the piezoelectric element to drive the piezoelectric element to cause a pump associated with the piezoelectric element to pump fluid.

FIGS. 12 and 13 are graphs of a pressure (P(t)) curve of the fluid pressure in the inflatable member as a function of time while the inflatable member is in an inflated state or configuration.

FIG. 14 is a flowchart of an example process of operating an implantable fluid-operated device.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

An implantable fluid-operated inflatable device may include a fluid control system. In some examples, the fluid control system includes at least one pump and/or at least one valve. In some examples, the components of the fluid control system control the flow of fluid between a fluid reservoir and an inflatable member of the implantable fluid-operated inflatable device, to provide for the inflation/pressurization and deflation/depressurization of the inflatable member. In some implementations, the fluid control system can be electronically-operated.

For example, the pumps and/or valves of the fluid control system can be electronically-operated by the fluid control system to control the pressure of, and the flow of fluid in, parts of the fluid-operated inflatable device. An electronically-operated fluid control system, in accordance with implementations described herein, can include a plurality of electromechanical devices, such as, piezoelectric devices that operate as pumps or as valves in the system. One or more processors or controllers can control the electromechanical devices. Additionally, the one or more processors, controllers, sensors or other devices, can monitor the status of the implantable device to detect misuse (such as by a patient) or other unintended or out of the ordinary uses. When a misused or other unintended use is detected, the one or more controllers can adjust the electronic control of the electromechanical device to facilitate continued operation of the electromechanical device and the safety of the patient in whom the inflatable device is implanted.

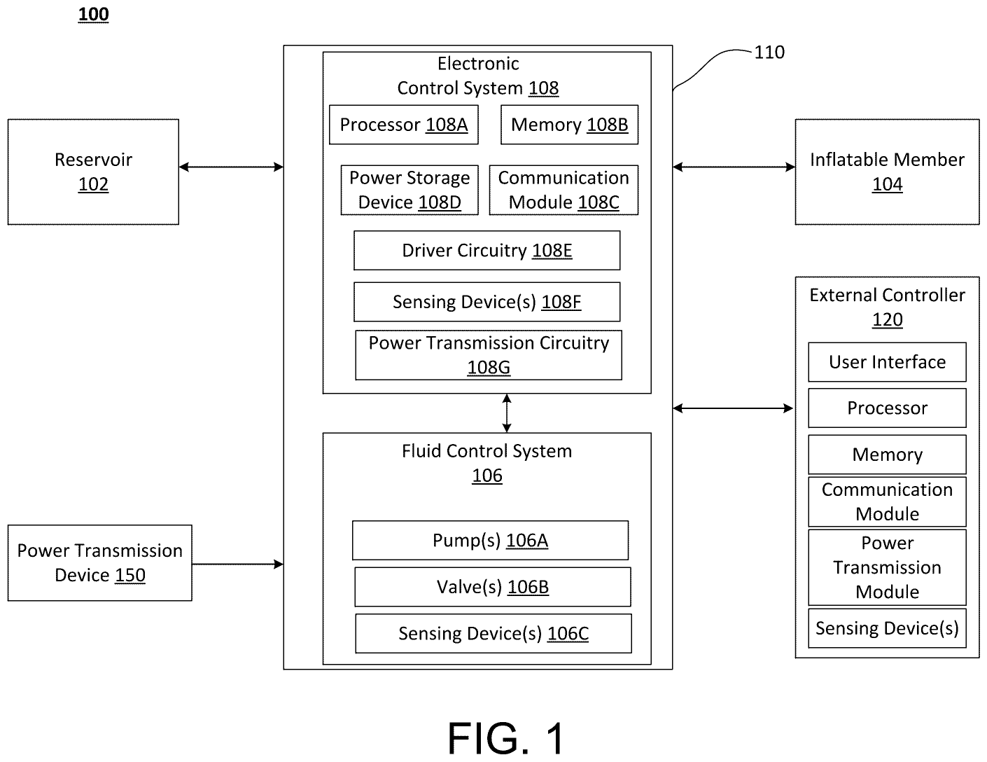

FIG. 1 is a block diagram of an example implantable fluid-operated inflatable device 100. The example inflatable device 100 shown in FIG. 1 includes a fluid reservoir 102, an inflatable member 104, and an electronic control system 108. The electronic control system 108 may interface with a fluid control system 106. The fluid control system 106 can include fluidics components such as one or more pumps 106A, one or more valves 106B and the like configured to transfer fluid between the fluid reservoir 102 and the inflatable member 104. The fluid control system 106 can include one or more sensing devices 106C, such as, for example, one or more pressure sensors, one or more flow rate sensors, etc., that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid-operated inflatable device 100 and/or communication with one or more external device(s). In some examples, the electronic control system 108 includes components such as a processor 108A, a memory 108B, a communication module 108C, a power storage device 108D (e.g., a battery), electronic driver circuity 108E, sensing devices 108F, such as, for example, voltage measurement circuitry, current measurement circuitry, an accelerometer, and other such components configured to provide for the monitoring, operation, and control of the implantable fluid-operated inflatable device 100, and power transmission circuitry 108G. In some examples, the communication module 108C of the electronic control system 108 may provide for communication with one or more external devices such as, for example, an external controller 120.

In some examples, the external controller 120 includes components such as, for example, a user interface, a processor, a memory, a communication module, a power transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation, and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, a technician, and the like.

In some examples, the power transmission module of the external controller 120 provides for charging of the components of the internal electronic control system 108. In some examples, transmission of power for the charging of the internal electronic control system 108 can be, alternatively or additionally, provided by an external power transmission device 150 that is separate from the external controller 120. In some implementations the external controller 120 can include sensing devices such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be internally implanted into the body of the patient. In some implementations, the electronic control system 108 and the fluid control system 106 are coupled in, or incorporated into, a housing 110. In some implementations, at least a portion of the electronic control system 108 is physically separate from the fluid control system 106. In some implementations, some modules of the electronic control system 108 are coupled to, or incorporated into, the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable fluid-operated inflatable device 100.

In some examples, electronic monitoring and control of the implantable fluid-operated inflatable device 100 may provide for the detection of misuse of the device (such as patient misuse) or other unintended uses of the device and adjustment of the device during such misuses or unintended uses to help prevent damage to the device and/or patient injury. For example, the fluidic architecture defining the flow and control of fluid through the implantable fluid-operated inflatable device 100, including the configuration and placement of fluidics components such as pumps, valves, sensing devices and the like, may allow the inflatable device 100 to precisely monitor and control operation of the inflatable device 100 to effectively respond to misuses or other unintended uses. In some implementations, the electronic monitoring and control of the inflatable device 100 may cause fluid to flow from the inflatable member 104 to the reservoir 102 when an excessive amount of pressure is detected in the inflatable member 104 due to, for example, a patient misuse of the device 100.

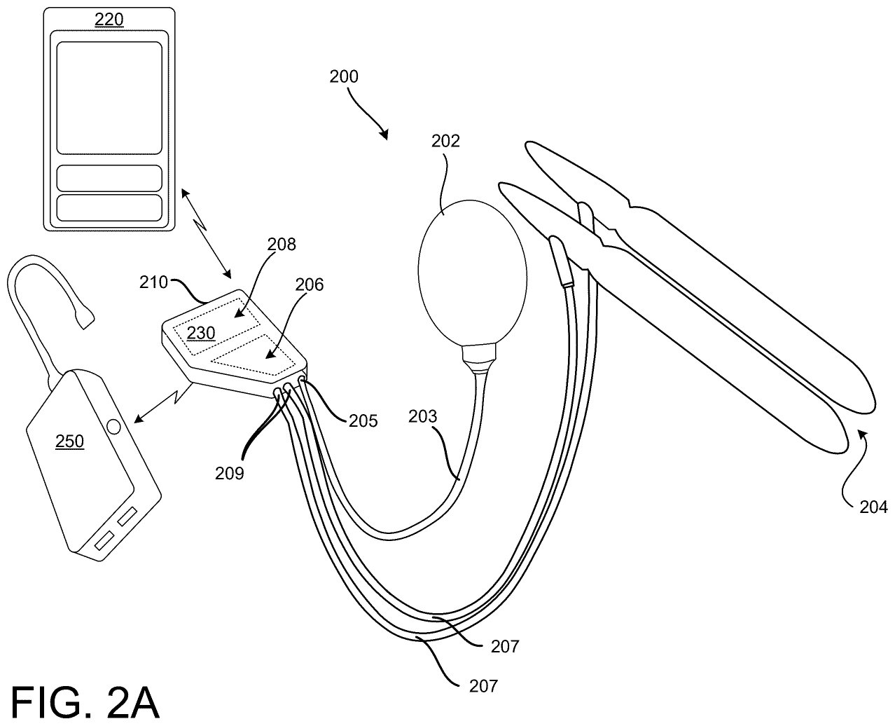

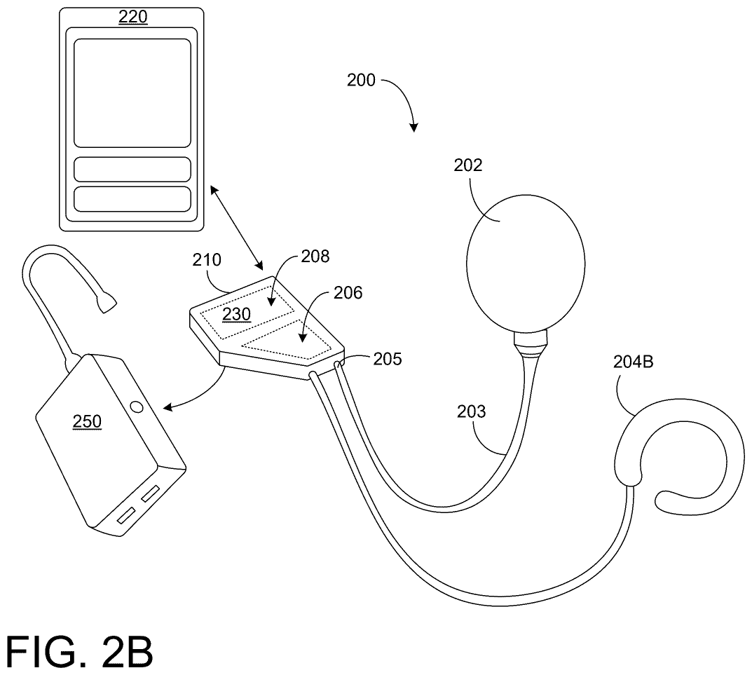

The example implantable fluid-operated inflatable device 100 may be representative of a number of different types of implantable fluid-operated devices. For example, the implantable fluid-operated inflatable device 100 shown in FIG. 1 may be representative of an inflatable penile prosthesis as shown in FIG. 2A. In another example, the implantable fluid-operated inflatable device 100 as shown in FIG. 2B may be representative of an artificial sphincter (such as an artificial urinary sphincter) that includes an inflatable member 204B that forms a loop or cuff. In some implementations, the example implantable fluid-operated inflatable device 100 shown in FIG. 1 may be representative of other types of implantable inflatable devices that rely on the control of fluid flow to components of the device to achieve inflation, pressurization, deflation, depressurization, deactivation, and the like.

An example system including an example implantable fluid-operated inflatable device 200 in the form of an example inflatable penile prosthesis is shown in FIG. 2A. The example inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes components such as, for example, one or more fluid control devices, one or more pressure sensors, and other such components. In some implementations, the example inflatable device 200 includes an electronic control system 208 (similar to the example electronic control system 108 described above with respect to FIG. 1) configured to provide for the transfer of fluid between a reservoir 202 (such as the example fluid reservoir 102 described above with respect to FIG. 1) and an inflatable member 204 (similar to the example inflatable member 104 described above with respect to FIG. 1) via the fluidics components. In the example shown in FIG. 2A, the inflatable member 204 is in the form of a pair of inflatable cylinders. In the example shown in FIG. 2A, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204.

In the example shown in FIG. 2A, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 209 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220 (similar to the external controller 120 described above with respect to FIG. 1), via respective communication modules. For example, an application stored in a memory and executed by a processor of the external controller 220 may allow the user and/or a physician to operate, view, monitor and alter operation of the inflatable device 200. In some examples, components of the electronic control system 208 and/or the fluid control system 206 can be charged and/or recharged by a power transmission module of the external controller 220, and/or by a power transmission device 250, that is separate from the external controller 220.

The principles to be described herein are applicable to the example implantable fluid-operated inflatable device, in the form of the example inflatable penile prostheses shown in FIG. 2A, and to other types of implantable fluid-operated inflatable devices that rely on pumps, valves and/or various fluidics components to provide for the transfer of fluid between the different fluid-filled implantable components to achieve inflation, deflation, pressurization, depressurization, deactivation, occlusion, and the like for effective operation. The example implantable fluid-operated inflatable device 200 shown in FIG. 2A includes an electronic control system 208 to provide for control of the operation of the respective inflatable members 204 in the form of cylinders, and the monitoring and control of pressure and/or fluid flow through inflatable members 204. Some of the principles to be described herein may also be applied to implantable fluid-operated inflatable devices that are manually controlled.

As noted above, the electronic control system 208 controlling the flow of fluid between the reservoir 202 and the inflatable member 204 for inflation, pressurization, deflation, depressurization and the like of the inflatable member 204 may provide for improved patient control of the inflatable device 200, improved accuracy in operation of the inflatable device 200, improved patient comfort, improved patient safety, and the like. In some situations, this improved control and improved accuracy in the operation of the inflatable device 200 may rely on precise operation and control of the components within the fluid control system 206 and/or the electronically controlled fluid manifold 230. Accordingly, in some implementations, the electronically controlled fluid manifold 230 includes a fluid control system 206 having one or more pump and one or more valve devices and one or more sensing devices. Accurate and consistent operation of the components of the pump and/or valve devices may produce the desired accurate flow control, and consistent inflation, deflation, pressurization, depressurization, deactivation, occlusion, and the like for effective operation.

A fluid control system, in accordance with implementations described herein, can include a pump assembly including, for example, one or more pump devices and valve devices within a fluid circuit of the pump assembly to control the transfer fluid between the fluid reservoir and the inflatable member. In some examples, the pump assembly including the one or more pump devices and valve device(s) is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump devices and valve devices include electric elements that are configured to be electronically actuated to change their shape and thereby to function as a pump or valve. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid-operated device to provide patient safety and device efficacy.

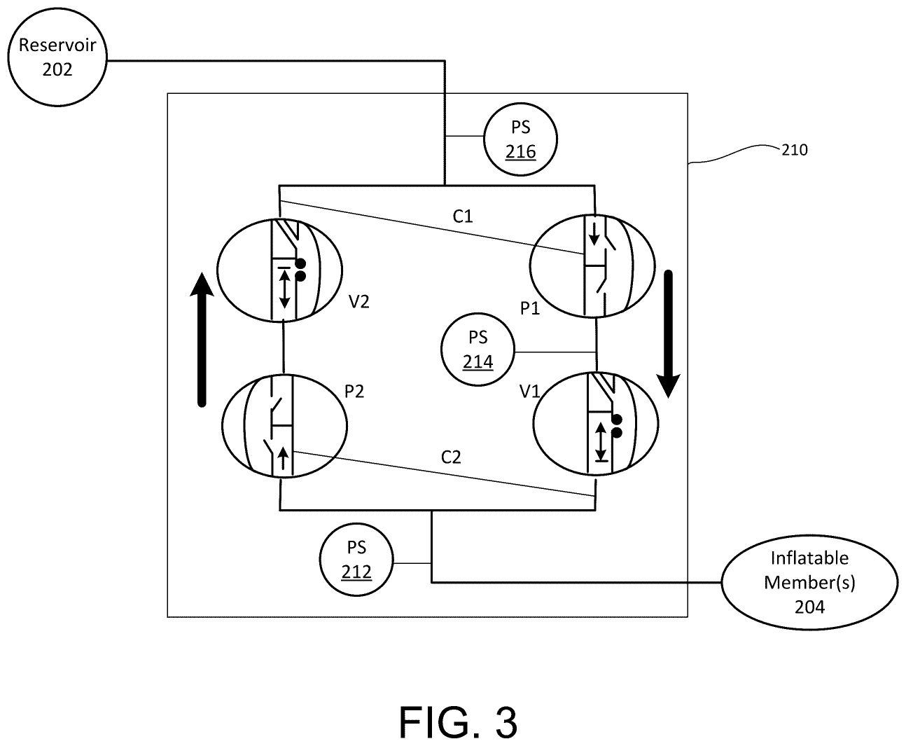

FIG. 3 is a schematic diagram of an example fluidic architecture for an electronically-operated implantable fluid-operated inflatable device, according to an aspect. The fluidic architecture of an implantable fluid-operated inflatable device can include other arrangements of fluidic passageways, pump(s)/valve(s), pressure sensor(s) and other components than the examples shown in FIG. 3.

The example fluidic architecture shown in FIG. 3 includes a first pump P1 and a first valve V1 positioned in a first fluid passageway, between the reservoir 202 and the inflatable member 204, to control the flow of fluid from the reservoir 202 to the inflatable member 204. The example fluidic architecture shown in FIG. 3 includes a second pump P2 and a second valve V2 positioned in a second fluid passageway, between the inflatable member 204 and the reservoir 202, to control the flow of fluid from the inflatable member 204 to the reservoir 202.

In example fluidic architecture shown in FIG. 3, the first pump P1 and the first valve V1 operate to pump fluid from the reservoir 202 to the inflatable member 204 through the first fluid passageway to provide for inflation of the inflatable member 204, while the second valve V2 closes the second fluid passageway to prevent backflow of fluid, back to the reservoir 202. The second pump P2 and the second valve V2 operate to pump fluid from the inflatable member 204 to the reservoir 202 through the second fluid passageway to provide for deflation of the inflatable member 204, while the first valve V1 closes the first fluid passageway to prevent backflow of fluid to the inflatable member 204.

In an example implementation, a conduit C1 can connect a section of the second fluid passageway that is downstream of pump P2 and valve V2 to a section of the first fluid passageway, for example, to an inlet portion of pump P1. Fluid flow through conduit C1 can flush fluid and material out from of the section of the first fluid passageway when fluid is pumped from the inflatable member 204 to the reservoir 202. In an example implementation, a conduit C2 can connect a section of the first fluid passageway that is downstream of pump P1 and valve V1 to a section of the second fluid passageway, for example, to an inlet portion of pump P2. Fluid flow through conduit C2 can flush fluid and material out from of the section of the second fluid passageway when fluid is pumped from the reservoir 202 to the inflatable member 204.

In some implementations, the example fluidic architecture can include one or more pressure sensors 212, 214, 216, each configured to measure a fluid pressure at a point in the system. For example, a first pressure sensor 212 can be connected to a fluidic passageway, conduit, chamber or component located fluidically between the inflatable member 204 and pumps P1, P2 and valves V1, V2, and can be configured to measure a fluid pressure at this location, which can also serve as a measure of a fluid pressure in the inflatable member(s) 204, because the fluid is essentially incompressible and the conduit between the pressure sensor 212 and the inflatable member(s) 204 can be considered to be free of obstruction. A second pressure sensor 214 can be connected to a fluidic passageway, conduit, chamber or component located fluidically between pump P1 and valve V1 and can be configured to measure a fluid pressure at this location. A third pressure sensor 216 can be connected to a fluidic passageway, conduit, chamber or component located fluidically between the reservoir 202 and pumps P1, P2 and valves V1, V2, and can be configured to measure a fluid pressure at this location, which can also serve as a measure of a fluid pressure in the reservoir, because the fluid is essentially incompressible and the conduit between the pressure sensor 216 and the reservoir 202 can be considered to be free of obstruction. In some implementations one or more of the pressure sensors 212, 214, 216 can be contained with the housing 210.

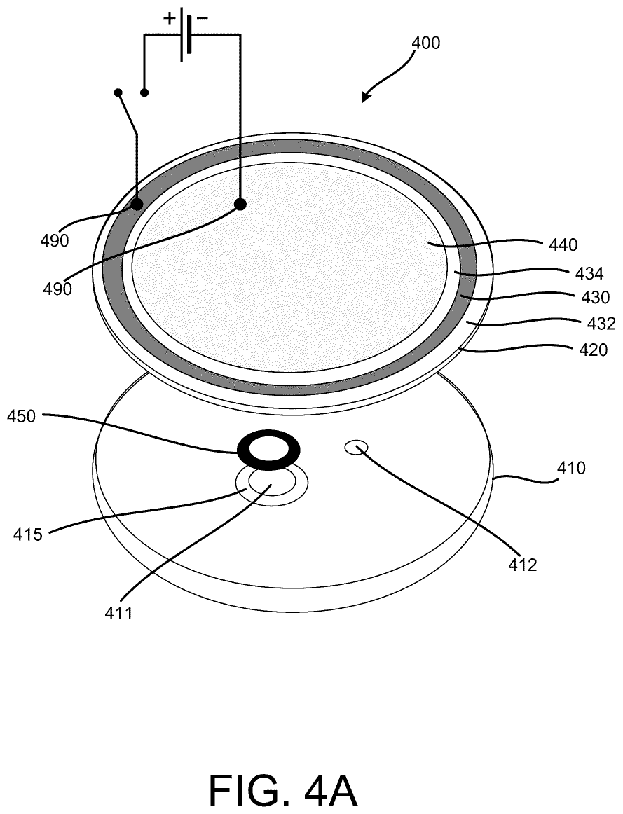

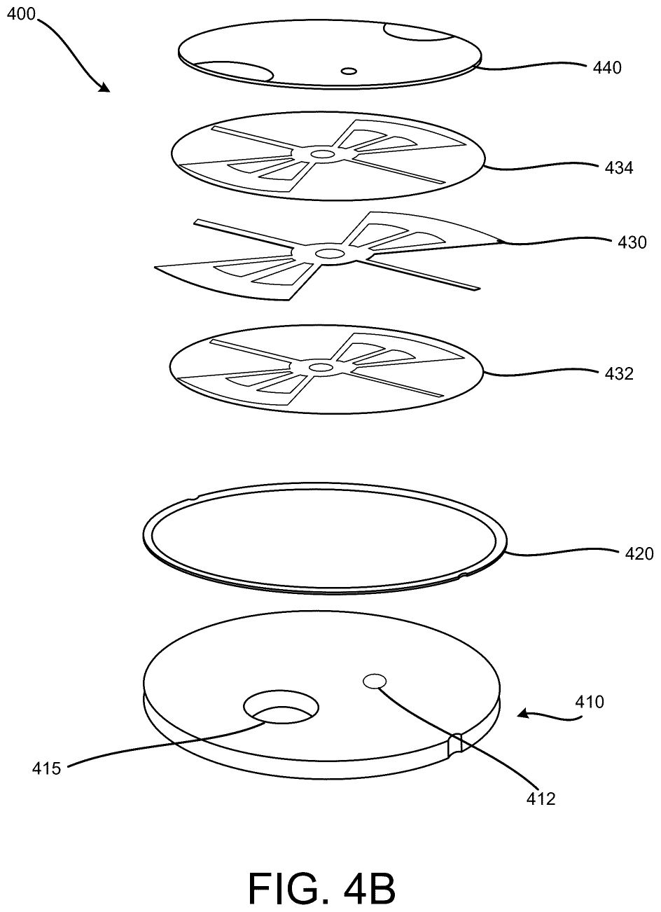

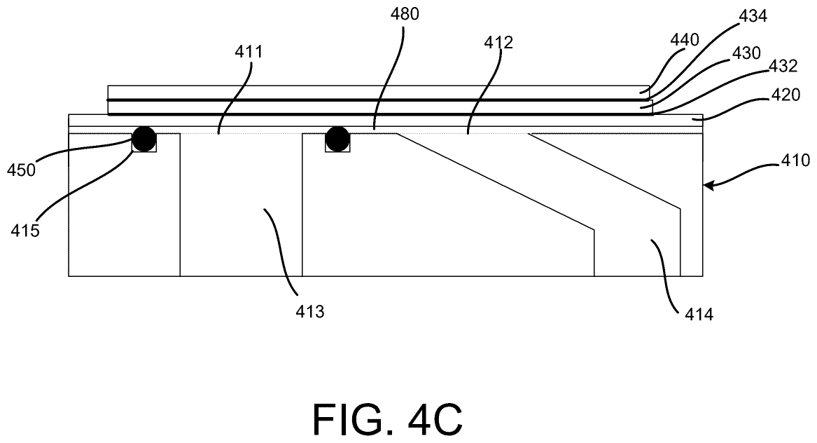

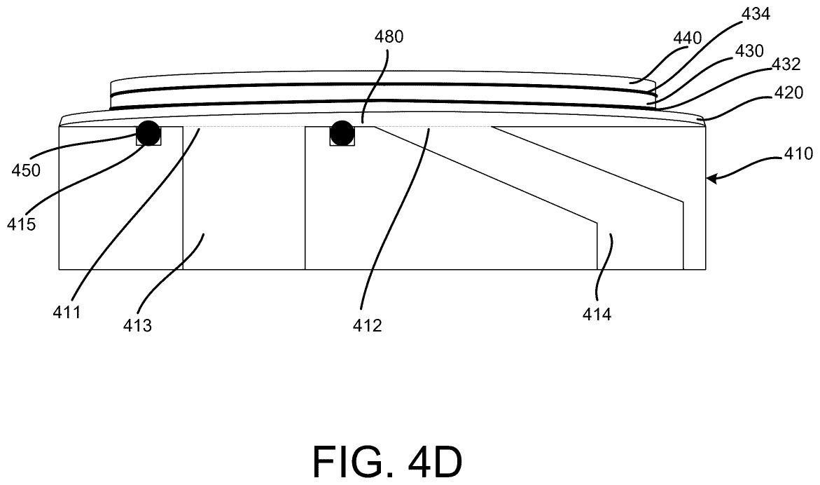

FIG. 4A is a partially exploded perspective view of an example valve device 400. FIG. 4B is an exploded perspective view of the example valve device 400. FIGS. 4C and 4D are cross-sectional views of the example valve device 400 shown in FIG. 4A, in an assembled state. The example valve device 400 shown in FIGS. 4A-4D is an example of a fluid control device, or a fluidic component, included in the fluid control system 206 of the example electronically controlled fluid manifold 230 described above.

In the example arrangement shown in FIGS. 4A-4D, the example valve device 400 includes a base plate 410 defining a base portion of the valve device 400. A diaphragm 420 is positioned on the base plate 410. A piezoelectric element 440 is positioned on the diaphragm 420, with an isolation layer 430 positioned between the diaphragm 420 and the piezoelectric element 440. The piezoelectric element can be electrically powered (e.g., by a battery of in the implantable fluid-operated inflatable device 100) to drive the diaphragm 420 to open and close the valve device 400. The diaphragm 420 can include a thin metal foil, whose shape can be repeatably deformed in response to movement by the piezoelectric element 440. In some implementations, the diaphragm 420 can include titanium material. In some implementations, the diaphragm 420 can include gold material. In some implementations, the diaphragm 420 can include stainless steel material or other alloys. In some implementations, the isolation layer 430 can include a polyamide material that has a high resistivity, for example, a resistivity greater than 10¹³ Ohm-cm to provide electrical isolation between the piezoelectric element 440 and the diaphragm 420.

In some examples, an epoxy layer 432 provides for the coupling of the isolation layer 430 and the diaphragm 420. In some examples, an epoxy layer 434 provides for the coupling of the piezoelectric element 440 and the isolation layer 430, and the epoxy layers 432, 434 together provide for the coupling of the piezoelectric element 440 to the diaphragm 420. In some implementations, the epoxy layers 432, 434 are not distinct but are part of one epoxy layer. The epoxy layers 432, 434 can be formed from a mixture of different chemicals (e.g., a resin and a hardener) that, when mixed and cured, react to form a covalent bond and that adhere to surfaces that they contact. Curing of the epoxy can be controlled through selection of the resin and hardener chemicals used in the mixture, selection of the ratio of the chemicals used in the mixture, control of the temperature of the mixture, and application of electromagnetic radiation to the mixture.

In some examples, one or more electrodes 490 are arranged on the example valve device 400. In the example shown in FIG. 4A, the example valve device 400 includes a pair of electrodes 490 coupled between the isolation layer 430 and the piezoelectric element 440. Application of a voltage to the piezoelectric element 440 causes a deflection or deformation of the piezoelectric element 440 and a corresponding deflection or deformation of the diaphragm 420 coupled thereto.

In the example arrangement shown in FIGS. 4A-4D, a fluid chamber 480 is defined between the base plate 410 and the diaphragm 420. For example, in some implementations, the diaphragm 420 can be bonded to the base plate 410 at the periphery of the diaphragm to form a fluid-tight connection between the base plate 410 and the diaphragm 420. The base plate 410 includes a first opening 411 that provides for communication between a first fluid passageway 413 and the fluid chamber 480. The base plate 410 includes a second opening 412 that provides for communication between a second fluid passageway 414 and the fluid chamber 480. In the example arrangement shown in FIGS. 4A-4D, the base plate 410 includes a recess 415 surrounding the first opening 411, with a seal 450, in the form of an O-ring in the example shown in FIGS. 4A-4D, fitted in the recess 415. In some examples, a top portion of the seal 450 is pressed against the diaphragm 420 in the closed position of the valve device 400, as shown in FIG. 4C to close off the chamber 480 and inhibit the flow of fluid through the example valve device 400, between the first fluid passageway 413 and the second fluid passageway 414 via the chamber 480. In some examples, in which the valve device 400 does not include a seal 450, the diaphragm 420 is seated against the base plate 410 to close off the chamber 480 and inhibit the flow of fluid through the valve device 400. In the open position of the example valve device 400, the base plate 410 and the top portion of the seal 450 are separated, or spaced apart from, the diaphragm 420 due to the deflection of the diaphragm 420. This positioning of the seal 450 and the base plate 410 relative to the diaphragm 420 opens the chamber 480 and allows fluid to flow through the example valve device 400, between the first fluid passageway 413 and the second fluid passageway 414 via the fluid chamber 480.

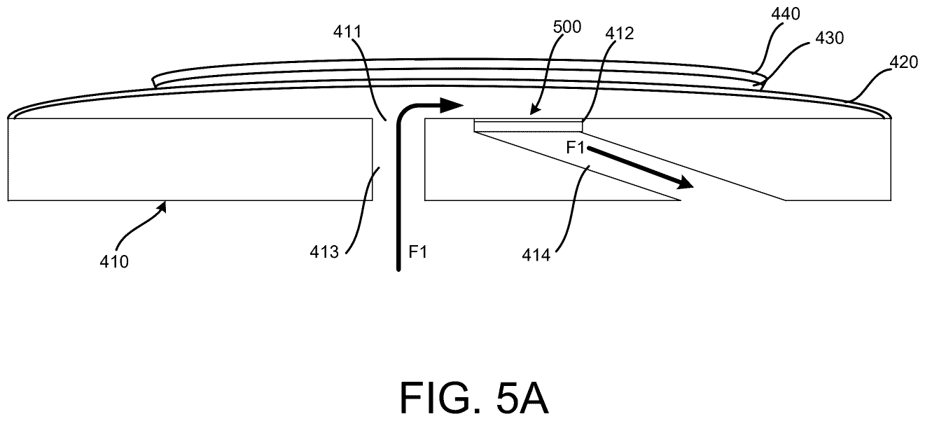

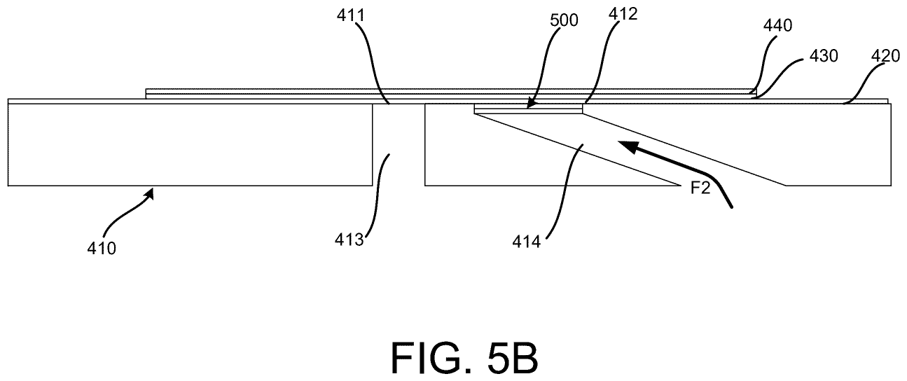

FIGS. 5A and 5B are cross-sectional views of the example valve device 400 shown in FIGS. 4A-4D, including an example flow control device 500 positioned in one of the fluid passageways of the example valve device 400.

FIG. 5A illustrates an example in which the valve device 400 is open, allowing fluid to flow in the direction of the arrows F1, through the first fluid passageway 413, into the chamber 480, and out of the valve device 400 through the second fluid passageway 414. The example shown in FIG. 5A may illustrate an open position of the valve device 400 that allows fluid to flow, for example, from the reservoir 202 to the inflatable member 204 to provide for inflation/pressurization of the inflatable member 204.

In the example arrangement shown in FIGS. 5A and 5B, the example flow control device 500 is positioned at the second opening 412 formed in the base plate 410, the second opening 412 providing for fluid communication between the fluid chamber 480 and the second fluid passageway 414. In some examples, the flow control device 500 is a check valve, or a one-way valve, that allows for flow in one direction (in this example, in the direction of the arrows F1), while inhibiting flow in the opposite direction.

FIG. 5B illustrates the closed position of the valve device 400, in which the flow of fluid through the valve device 400 is blocked. In some examples, the closed position shown in FIG. 5B may maintain an inflation pressure of the inflatable member 204. As described above, in some situations, pressure fluctuations and/or pressure spikes may exert a force, or pressure on the valve device 400 in the closed position. FIG. 5B illustrates a pressure spike, or a back pressure, exerted in the direction of the arrow F2. In the example described above with respect to FIGS. 4A-4D, this type of pressure spike, or back pressure exerted on the diaphragm 420/piezoelectric element 440 could cause an unintentional opening of the valve device 400, and an unintentional deflation/depressurization of the inflatable member 204. In the example shown in FIG. 5B, the flow control device 500 (positioned at the second opening 412, between the second fluid passageway 414 and the fluid chamber 480), for example, in the form of a check valve or a one-way valve, remains in the closed position in response to the pressure spike/back pressure/flow of fluid in the direction of the arrow F2. Thus, the positioning of the flow control device 500 at the second opening 412, allowing flow in a first direction, i.e., the direction of the arrows F1, while blocking flow in a second direction, i.e., the direction of the arrow F2, maintains the closed state of the valve device 400, even in response to fluctuation in pressure, or pressure spike, or back pressure.

The general architecture and principles of operation of the valve device described above also can be used to implement one or more pumps (such as pumps that pumps P1, P2 of FIG. 3) to pump fluid from one location to another. For example, repeated movement of a diaphragm between an open position and a closed position, relative to a base plate, can cause fluid to be drawn into a chamber formed between the diaphragm and the base plate through a first fluid passageway and expelled out of the chamber into a second fluid passageway. In this manner, fluid can be pumped from a first location that is fluidically connected to the first passageway to a second location that is fluidically connected to the second passageway. In some implementations, one or more one-way valves can be configured to prevent, or limit, the flow of fluid in the direction from the second location to the first location.

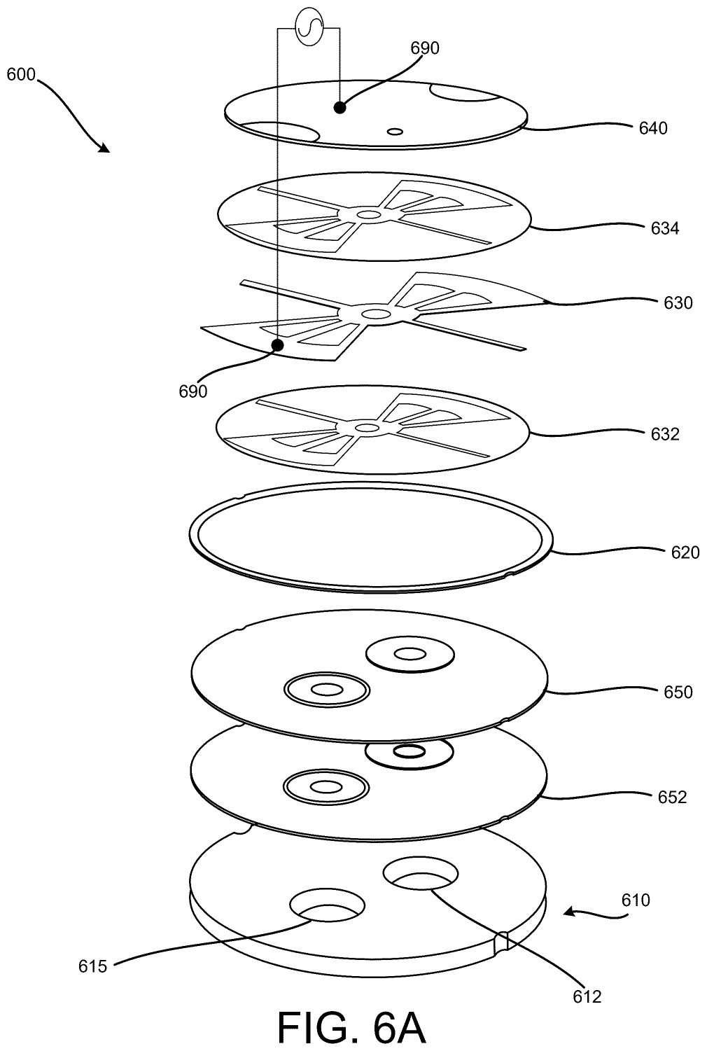

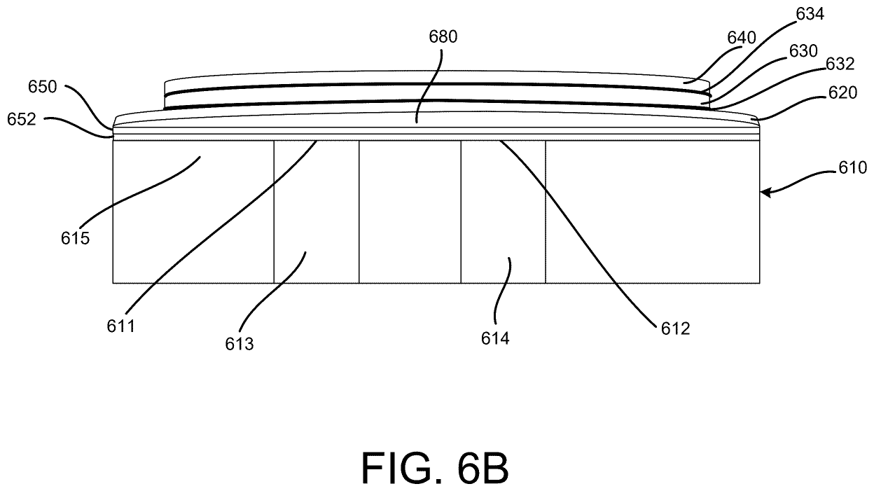

FIG. 6A is a partially exploded perspective view of an example pump device 600, and FIG. 6B is a cross-sectional view of the example pump device 600. The example pump device 600 shown in FIGS. 6A-6B is an example of a fluid control device, or a fluidic component, included in the fluid control system 206 of the example electronically controlled fluid manifold 230 described above.

In the example arrangement shown in FIGS. 6A-6B, the example pump device 600 includes a base plate 610 defining a base portion of the pump device 600. A diaphragm 620 is positioned on the base plate 610. A piezoelectric element 640 is positioned on the diaphragm 620, with an isolation layer 630 positioned between the diaphragm 620 and the piezoelectric element 640. The piezoelectric element can be electrically powered (e.g., by a battery of the implantable fluid-operated inflatable device 100) to drive the diaphragm 620 to pump fluid through the pump device 600. The diaphragm 620 can include a thin metal foil, whose shape can be repeatably deformed in response to movement by the piezoelectric element 640. In some implementations, the diaphragm 620 can include titanium material. In some implementations, the diaphragm 620 can include gold material. In some implementations, the diaphragm 620 can include stainless steel material or other alloys. In some implementations, the isolation layer 630 can include a polyamide material that has a high resistivity, for example, a resistivity greater than 10¹³ Ohm-cm to provide electrical isolation between the piezoelectric element 640 and the diaphragm 620.

In some examples, an epoxy layer 632 provides for the coupling of the isolation layer 630 and the diaphragm 620. In some examples, an epoxy layer 634 provides for the coupling of the piezoelectric element 640 and the isolation layer 630, and the epoxy layers 632, 634 together provide for the coupling of the piezoelectric element 640 to the diaphragm 620. In some implementations, the epoxy layers 632, 634 are not distinct but are part of one epoxy layer. The epoxy layers 632, 634 can be formed from a mixture of different chemicals (e.g., a resin and a hardener) that, when mixed and cured, react to form a covalent bond and that adhere to surfaces that they contact. Curing of the epoxy can be controlled through selection of the resin and hardener chemicals used in the mixture, selection of the ratio of the chemicals used in the mixture, control of the temperature of the mixture, and application of electromagnetic radiation to the mixture.

In some examples, one or more electrodes 690 are arranged on the example pump device 600. In the example shown in FIG. 6A, the example pump device 600 includes a pair of electrodes 690 coupled between the isolation layer 630 and the piezoelectric element 640. Application of a voltage to the piezoelectric element 640 causes a deflection or deformation of the piezoelectric element 640 and a corresponding deflection or deformation of the diaphragm 620 coupled thereto.

When the pump device 600 is used in the fluid control system 206 of the example electronically controlled fluid manifold 230 described above, the piezoelectric element 640 can be controlled to cause fluid to be pumped by device 600, for example, by repeatedly changing a volume of the fluid chamber 680 by deforming the deformable diaphragm 620 to pump fluid from the fluid reservoir to the inflatable member.

In the example arrangement shown in FIGS. 6A-6B, a fluid chamber 680 is defined between the base plate 610 and the diaphragm 620. The base plate 610 includes a first opening 615 that provides for communication between a first fluid passageway 613 and the fluid chamber 680. The base plate 610 includes a second opening 612 that provides for communication between a second fluid passageway 614 and the fluid chamber 680. In some examples, the diaphragm 620 can be actuated to move between a closed position in which the diaphragm 620 is proximate to the base plate 610 due to the deflection of the diaphragm 620, such that the volume of the chamber 680 is minimized, and an open position in which the base plate 610 is separated, or spaced apart from, the diaphragm 620 due to the deflection of the diaphragm 620, such that the volume of the chamber is maximized. When the diaphragm 620 is actuated to move from the closed position to the open position, fluid can be drawn into the chamber 680 through the first fluid passageway 613, and when the diaphragm 620 is actuated to move from the open position to the closed position, fluid can be expelled from the chamber 680 through the second fluid passageway 614. Repeatedly actuating the diaphragm between the closed and open position allows fluid to be pumped through the pump device 600, from the first fluid passageway 613 to the second fluid passageway 614 via the fluid chamber 680.

In some implementations, the pump device 600 can include one or more foil plates 650 and 652 to control the flow of fluid into and out of the pump device 600. The foil plates 650, 652 can include one-way check valves that operate to permit fluid to flow in one direction through the values but not in an opposite direction. The one-way check valves defined by the one or more foil plates can be positioned in, or in fluid connection with, a fluid passageway 613, 614 of the pump device 600. In some examples, a check valve is positioned in, or in fluid connection with, a portion of a fluid passageway 613, 614 so as to inhibit the unintended flow of fluid through the pump device in the event of a fluctuation, or spike in pressure. In some examples, a check valve is positioned in a fluid passageway 613, 614 so as to counteract a back pressure that would otherwise overcome the closing pressure and cause unintentional flow through the pump device 600. In some example implementations, a first check valve defined by one or more foil plates 650, 652 is positioned in, or in fluid connection with (e.g., at a first opening 611 of), a first fluid passageway 613 of the pump device and is configured to permit fluid to easily flow from the first fluid passageway 613 into the chamber 680 but to prevent or inhibit the flow of fluid from the chamber 680 into the passageway 613. In some example implementations, a second check valve defined by one or more foil plates 650, 652 is positioned in, or in fluid connection with (e.g., at a first opening 612 of), a second fluid passageway 614 of the pump device 600 and is configured to permit fluid to easily flow from the chamber 680 into the second fluid passageway 613 but to prevent or inhibit the flow of fluid from the passageway 613 into the chamber 680.

Application of an alternating current (AC) voltage to the piezoelectric element 640 can cause the diaphragm 620 of the pump device 600 to oscillate between a first position that defines the closed position of the chamber 680, in which the diaphragm 620 is proximate to the base plate 610 and the volume of the chamber 680 is minimized, and a second (e.g., domed) position that defines the open position of the chamber 680, in which the diaphragm 620 is separated from the base plate and the volume of the chamber 680 is maximized. As the diaphragm 620 of the pump device 600 oscillates between a first position and the second position, fluid is drawn into the chamber 680 from the first passageway 613 and is expelled from the chamber 680 into the second passageway 614. As the diaphragm 620 of the pump device 600 oscillates between a first position and the second position, the one-way check valves defined by the one or more foil plates 650, 652 prevent or inhibit fluid from flowing from the chamber 680 into the first passageway 613 and prevent or inhibit fluid from flowing into the chamber 680 from the second passageway 614. Thus, the application of the AC voltage to the piezoelectric element 640 causes the pump device 600 to pump fluid from the first passageway 613 to the second passageway 614.

The frequency of the AC voltage applied to the piezoelectric element 640 can determine an oscillation mode of the piezoelectric element 640. In some implementations, the frequency of the AC voltage is selected to excite a lowest-order mode in which the center of the circular piezoelectric element 640 experiences the greatest extent of movement during an oscillation cycle, such that an amount of fluid pumped during an oscillation cycle is maximized compared to other oscillation modes.

The piezoelectric element 640 can be controlled to cause fluid to be pumped by device 600, for example, by repeatedly changing a volume of the fluid chamber 680 by deforming the deformable diaphragm 620 to pump fluid from the fluid reservoir to the inflatable member.

The volume of the chamber 680 can be determined, at least in part, by the shape, geometry, and material properties of the components used to form the chamber 680, including, for example, the base plate 610 and the deformable diaphragm 620. In some cases, a relatively larger volume of the chamber 680, for an approximately constant diameter of the chamber, can result in more fluid being pumped in each open/close cycle of the pump 600. To achieve a relatively larger volume of chamber 680, the deformable diaphragm can be deformed or biased into a non-flat dome-shaped configuration before it is attached to the piezoelectric element 640.

In some implementations, before the diaphragm 620 is placed in attached to the piezoelectric element 640, a voltage can be placed across the electrodes 690 attached to the piezoelectric element 640 to configure the piezoelectric element 640 in the domed configuration that is assumes when the fluid chamber is in the open position (See FIG. 4D). Then, the diaphragm can be placed in contact with the piezoelectric element while the piezoelectric element 440 is in its domed configuration, and the epoxy can be cured when the piezoelectric element and the diaphragm 420 are in the domed configuration, which can reduce stress on the adhesive bond between the diaphragm 420 and the piezoelectric element 440.

Referring again to FIG. 2A, although considerable effort is expended to maintain the cleanliness of the components of the system and the purity of the fluid used within the system, it is still possible that some small amounts of foreign matter can contaminate the fluid within the system. For example, when the reservoir 202, the inflatable members 204, and the housing 210 are implanted and connected (e.g., by conduits 203, 207) within a patient, it is possible that some contamination enters the fluidic system. In addition, it is possible that, once implanted within a patient, that small amounts of material disintegrate from walls of the reservoir 202, inflatable member 204, housing 210 and conduits 203, 207 and become suspended within fluid that flows within the inflatable device 200. Because of the small internal dimensions of the pumps and valves used within the fluidic system, the existence of particles of foreign matter suspended within the fluid flowing within the system poses a risk of clogging or damaging one or more of the pumps and valves, which may lead to malfunction of the inflatable device 200. To mitigate the effect of any particulate matter suspended within the fluid that flows within the inflatable device 200, the fluidic path can include one or more filters that block, or reduce the amount of, particulate matter that enters the pumps and valves of the system. In some implementations, the filters can be included in a fluid pathway of a pump or valve.

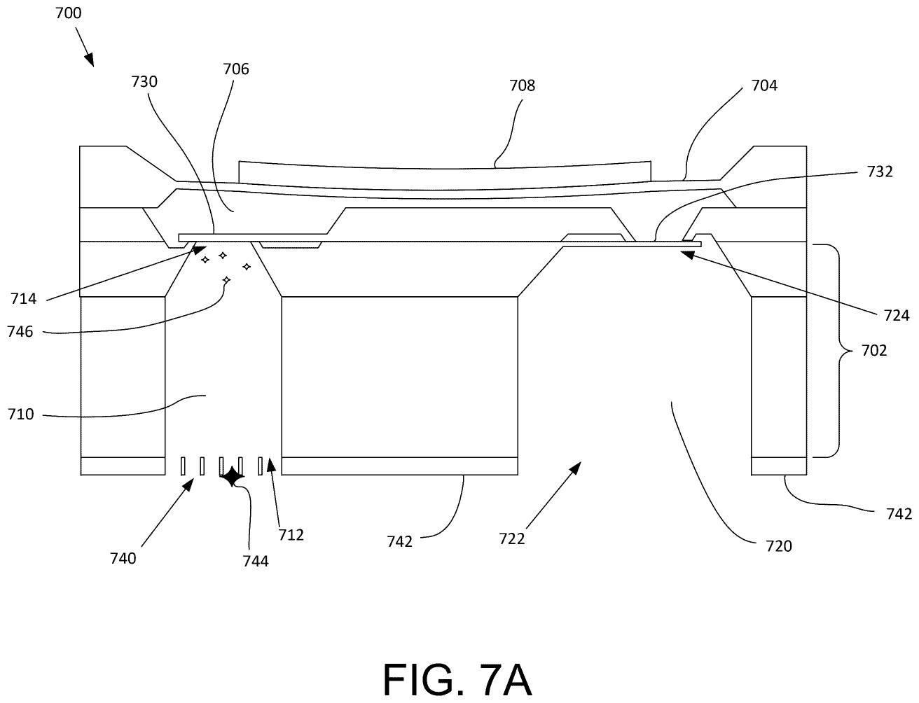

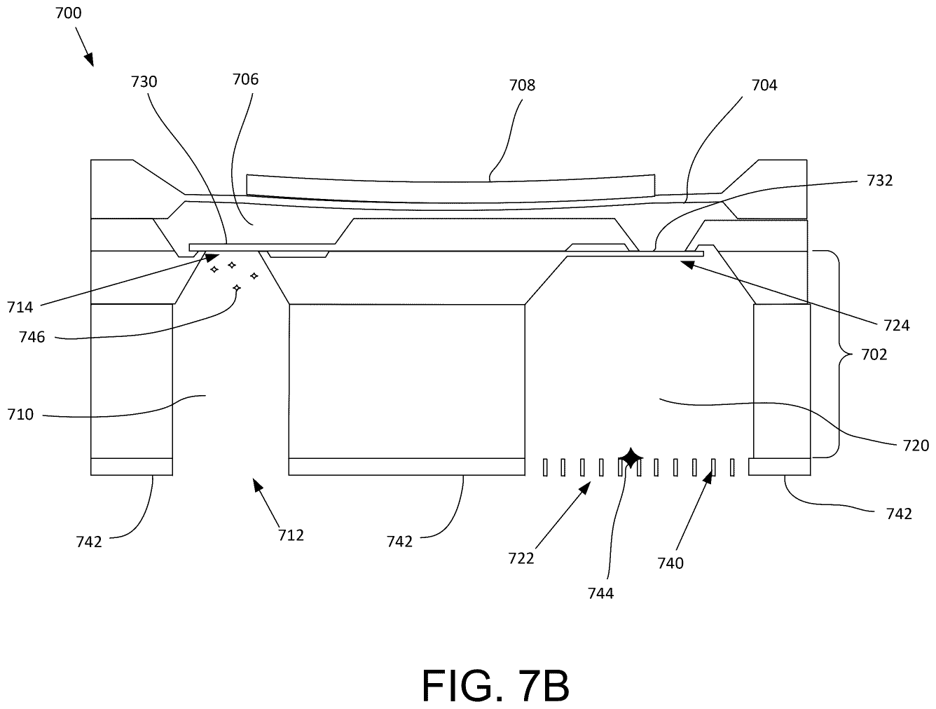

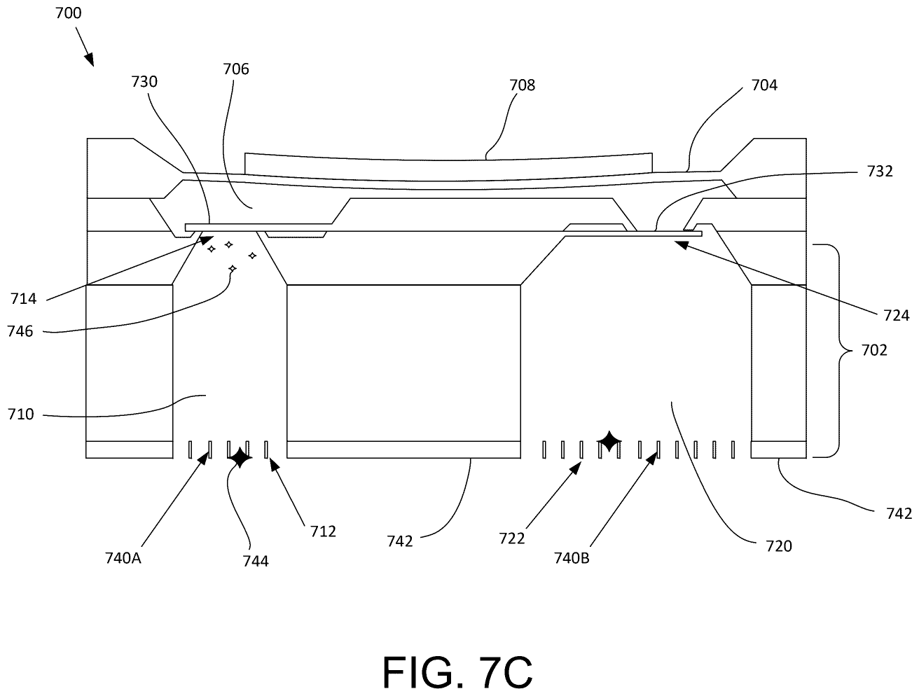

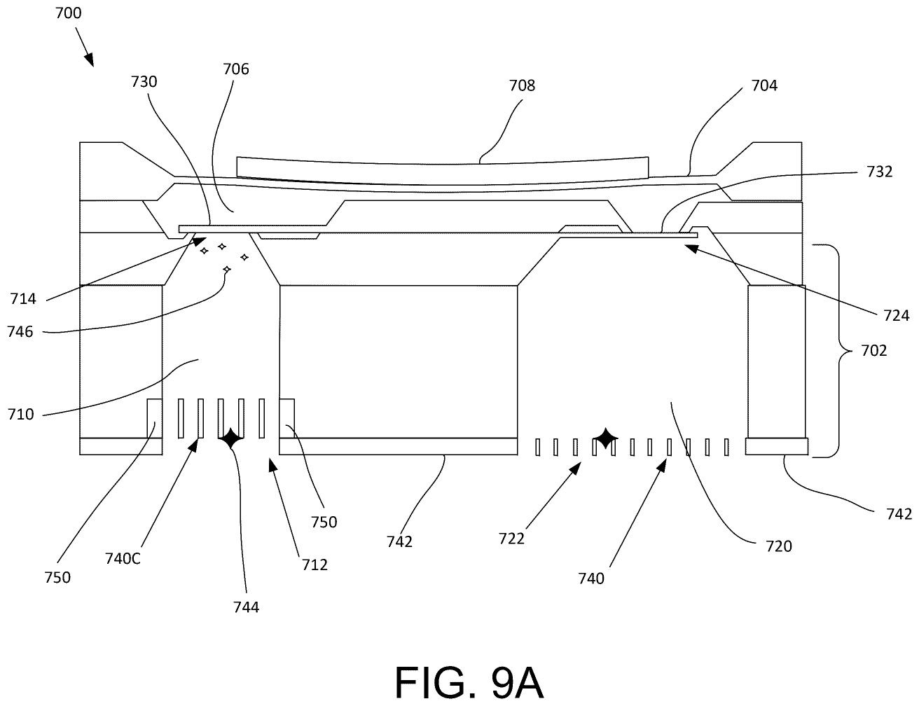

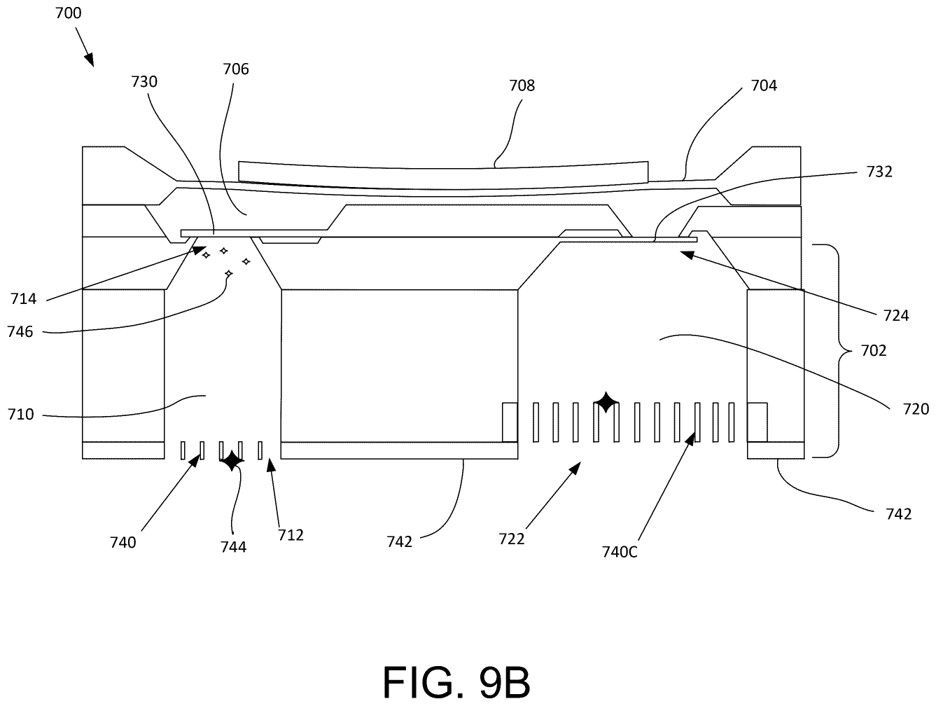

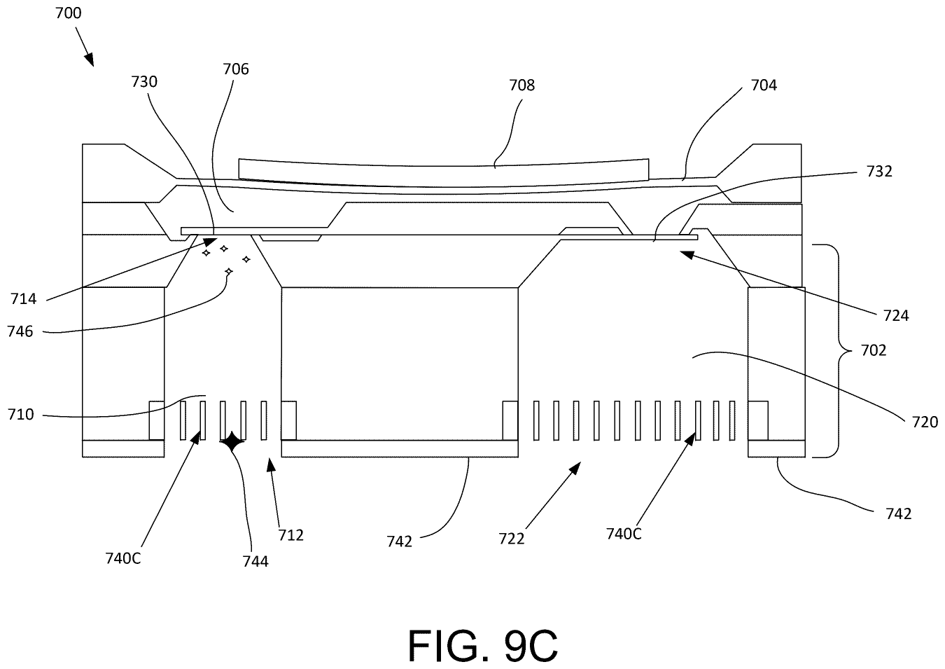

FIGS. 7A, 7B, 7C, 9A, 9B, and 9C are cross-sectional views of example pump devices 700 that includes a filter for capturing particulate matter in the fluid flow and/or for blocking the particulate matter from entering certain parts of the fluidic system (e.g., for blocking particulate matter from entering a pump chamber of the device). The example pump device 700 shown in FIGS. 7A, 7B, 7C, 9A, 9B, and 9C are examples of a fluid control device, or a fluidic component, included in the fluid control system 206 of the example electronically controlled fluid manifold 230 described above.

In the example arrangements shown in FIGS. 7A, 7B, 7C, 9A, 9B, and 9C, the example pump device 700 includes a base plate 702 defining a base portion of the pump device 700. A diaphragm 704 is positioned above the base plate 702, and a fluid chamber 706 is defined between the base plate 702 and the diaphragm 704. A piezoelectric element 708 is positioned on the diaphragm 704. The piezoelectric element can be electrically powered (e.g., by a battery of the implantable fluid-operated inflatable device) to drive the diaphragm 704 to pump fluid through the pump device 700. The diaphragm 704 can include a thin metal foil, whose shape can be repeatably deformed in response to movement by the piezoelectric element 708. In some implementations, the diaphragm 704 can include titanium material.

The base plate 702 can define a first fluid passageway 710 through which fluid can flow from a fluid reservoir into the fluid chamber 706. The first fluid passageway 710 can include an opening 712 at a first end of the passageway 710, which is distal to the fluid chamber 706, and can include an opening 714 and a second end of the passageway 710, which is proximate to the fluid chamber 706. The base plate 702 can define a second fluid passageway 720 through which fluid can flow from the fluid chamber 706 to an inflatable member. The second fluid passageway 720 can include an opening 722 at a first end of the passageway 720, which is distal to the fluid chamber 706, and can include an opening 724 and a second end of the passageway 720, which is proximate to the fluid chamber 706. In some implementations, the first fluid passageway 710 and the second fluid passageway 720 can be tapered, such the passageways 710, 720 have larger cross-sectional areas at the ends 712, 722 of the passageways that are distal to the fluid chamber 706 than at ends of the passageways that are proximate to the fluid chamber.

The pump device 700 can include a first flexible flap 730 that includes a portion that has an area that is greater than an area of the passageway opening 714 that is proximate to the fluid chamber 706 and that covers the opening, such that the first flexible flap 730 is configured to seal against portions of the base plate that defines the opening 714 of the first fluid passageway 710 to close the opening 714 when a fluid pressure in the fluid chamber 706 is greater than a fluid pressure of fluid in the first fluid passageway 710. The flexible flap 730 can be secured to the base plate over a portion of its extent but can have a portion that is unsecured, such that at least a portion of the flexible flap is configured to be pushed away from one or more walls of the fluid passageway 710 that defines the opening 714 when a fluid pressure of fluid in the first fluid passageway 710 is greater than a fluid pressure in the fluid chamber 706. In this manner, the flexible flap 730 operates to allow fluid to flow from the first fluid passageway 710 into the fluid chamber 706 but to block the flow of fluid from the fluid chamber 706 into the first fluid passageway 710. The flexible flap 730 can be made of a variety of materials including, for example, titanium, elastomeric material, plastic material, etc.

The pump device 700 can include a second flexible flap 732 that includes a portion that has an area that is greater than an area of the passageway opening 724 that is proximate to the fluid chamber 706 and that covers the opening, such that the second flexible flap 732 is configured to seal against portions of the base plate that defines the opening 724 of the second fluid passageway 720 to close the opening 724 when a fluid pressure in the fluid chamber 706 is greater than a fluid pressure of fluid in the second fluid passageway 720. The flexible flap 732 can be secured to the base plate over a portion of its extent but can have a portion that is unsecured, such that at least a portion of the flexible flap is configured to be pushed away from one or more walls of the second fluid passageway 720 that defines the opening 724 when a fluid pressure in the fluid chamber 706 is greater than a fluid pressure of fluid in the second fluid passageway 720. In this manner, the flexible flap 732 operates to allow fluid to flow from the fluid chamber 706 into the second fluid passageway 720 but to block the flow of fluid from the second fluid passageway 720 into the fluid chamber 706. The flexible flap 732 can be made of a variety of materials including, for example, titanium, elastomeric material, plastic material, etc.

With the flexible flaps 730, 732 configured in this way to allow fluid to flow in a first direction from the first fluid passageway 710 into the fluid chamber 706 and out of the fluid chamber into the second fluid passageway 720 but not in a direction opposite to the first direction, repeated expansion and contraction of the volume of the fluid chamber 706 in response to the piezoelectric element 708 operating on the deformable diaphragm 704 can cause fluid to be pumped from a reservoir fluidically connected to the first fluid passageway 710 to an inflatable member that is fluidically connected to the second fluid passageway 720.

The pump device 700 can include a fluid filter 740 that is located within, or at the end 712 of, the first fluid passageway 710 or that is located within, or at the end 722 of, the second fluid passageway 720. The fluid filter 740 can operate to block, for example, debris, foreign matter, particulates suspended in the fluid flowing through the device 700 from passing through the first fluid passageway 710 and into the fluid chamber 706 and/or from exiting the second fluid passageway 720. For example, as shown in FIG. 7A, a fluid filter 740 is located at the opening 712 into the first fluid passageway 710. As shown in FIG. 7B, a fluid filter 740 is located at the opening 722 into the second fluid passageway 720. As shown in FIG. 7C, a fluid filter 740A is located at the opening 712 into the first fluid passageway 710, and a fluid filter 740B is located at the opening 722 into the second fluid passageway 720.

In some implementations, the fluid filter 740, 740A, 740B can include a metal foil (e.g., a titanium foil, having a pattern of openings that permit fluid to flow through the openings but that block particulates having a characteristic size larger than a threshold size from flowing through the opening. For example, particulates 744 having a characteristic size (e.g., minimum transverse extent) that is greater than a threshold size defined by the size (e.g., diameter) of the openings can be blocked by the filter 740, while particulates 746 and a characteristic size smaller than the threshold size can pass through the filter 740.

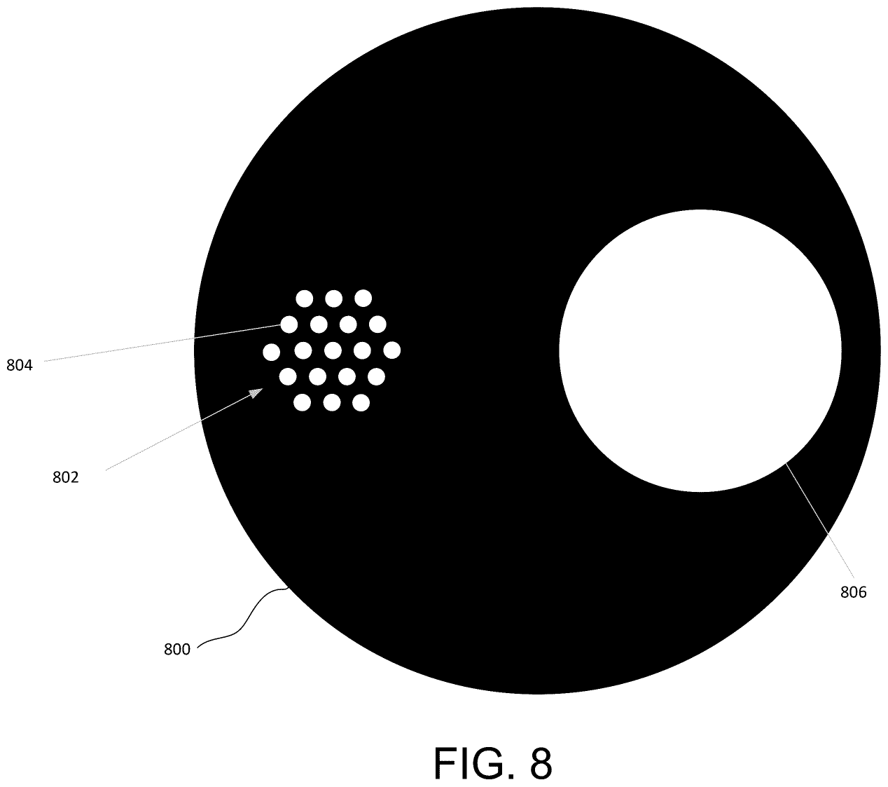

FIG. 8 is a schematic end view of a filter foil 800. In some implementations, the filter foil 800 can be made of metal (e.g., titanium) and can have a first section 802 that includes a plurality of openings 804. The openings can have a variety of different shapes, including circular, oblong, square, rectangular, hexagonal, etc. The plurality of openings 804 can be arranged in a regular or irregular pattern. For example, the openings 804 can be arranged in a two-dimensional hexagonal pattern, as shown in FIG. 8, or in a square pattern, or another type of regular or irregular pattern.

The plurality of openings 804 can be formed in the filter foil 800 in a number of different ways. For example, in some implementations, the pattern of openings can be mechanically stamped into the metal foil 800. In some implementations, the pattern of openings 804 can be laser etched into the metal foil 800. In some implementations, the pattern of openings can be chemically etched (e.g., through a lithographic process) into the metal foil 800.

Referring again to FIG. 7A and also to FIG. 8, the section 802 that includes the plurality of openings 804 can be arranged on the filter foil 800 so that the pattern of openings 804 is aligned with the opening 712 of the first fluid passageway 710 when the filter foil 800 is attached to the base plate 702. The filter foil 800 also can include an opening 806 in the filter foil that is aligned with the opening 722 of the second fluid passageway 720 of the base plate 702 when the filter foil is attached to the base plate.

In some implementations, the filter foil 800 can be welded to the base plate 702. For example, when the base plate includes titanium and the filter foil 800 includes titanium, the filter foil 800 can be welded to the titanium base plate 702. Prior to attaching (e.g., welding) the filter foil 800 to the base plate 702, the filter foil 800 can be positioned relative to the openings 712, 720 in the base plate, such that the first section 802 of the filter foil, which includes the plurality of openings 804, is positioned at the end of the first fluid passageway 710 and such that the opening 806 in the filter foil 800 is positioned at the end of the second fluid passageway 720. Similarly, when a filter foil is attached to the base plate shown in FIG. 7B, a section of the filter foil having a plurality of openings can be aligned with the end of the second fluid passageway 720, and a larger opening in the filter foil 800 in the aligned with the end of the first fluid passageway 710. Similarly, when a filter foil is attached to the base plate shown in FIG. 7C, a first section having a plurality of openings can be aligned with the end of the second fluid passageway 720 and a second section having a plurality of openings can be aligned with the end of the first fluid passageway 710.

In implementations in which the first fluid passageway 710 and the second fluid passageway 720 are tapered, such the passageways 710, 720 have larger cross-sectional areas at the ends 712, 722 of the passageways that are distal to the fluid chamber 706 than at ends of the passageways that are proximate to the fluid chamber, filters 740, 740A, 740B positioned at the distal ends of the fluid passageways 710, 720 can have cross-sectional areas that are greater than the cross-sectional areas of the openings 714, 724 between the passageways 710, 720 and the fluid chamber 706. Because of this the area of the filter that is active for trapping particulate matter can be larger than the areas of the openings 714, 724 between the passageways 710, 720 and the fluid chamber 706. In some implementations the flow of fluid through the filter 740, 740A, 740B can be reversed to dislodge some of the particulate matter that has been trapped by the filters from the filters.

For example, referring again to FIG. 3, a fluid conduit C1 can be provided between a downstream side of valve V2 and a pump P1. When the pump P1 is configured similarly to the pump shown in FIG. 7A, with a filter 740 at the end of the fluid passageway 710, the fluid conduit C1 can be connect to the first fluid passageway 710, so that when fluid is pumped from inflatable member(s) 204 to the reservoir 202 some of that fluid is pumped into the fluid passageway 710 of pump P1. Then, with valve V1 closed, the fluid that enters the first fluid passageway 710 of pump P1 can flow out of the distal end 712 of the first fluid passageway 710 and back to the reservoir 202. The fluid that flows out of the distal end 712 of the first fluid passageway 710 can flush debris and particulate matter out of the filter 740. In some implementations, the conduit C1 can include a one-way valve that allow fluid to pass from valve V2 to pump P1 but not in the opposite direction. Other such fluid connections, for example, conduit C2 of FIG. 3, can be used to flush debris and particulate matter out of filters used in the fluid control system.

The example pump devices 700 shown in FIGS. 7A, 7B, 7C include filters 740, 740C for blocking particulate matter in the fluid from entering a pump chamber of the device or for circulating in the fluidic system in which the pump devices operate. The filter 740 shown in FIG. 7A is disposed at the distal end 712 of the first fluid passageway 710, and the filter 740 shown in FIG. 7B is disposed at the distal end 722 of the second fluid passageway 720. These filters 740 can include a plurality of openings in a foil, where the size of the openings is selected to block the passage of particles having a characteristic size greater than a threshold size and to allow fluid and particles having a characteristic size less than the threshold size to pass through the openings.

In some implementations, the example pump devices 700 shown in FIGS. 7A, 7B, 7C can include filters 740C disposed within the first fluid passageway 710 or within the second fluid passageway 720, for example, between the first end 712 of the first fluid passageway 710 and the opening 714 at the second end of the first fluid passageway 710 and/or between the first end 722 of the second fluid passageway 720 and the opening 724 at the second end of the second fluid passageway 720. For example, as shown in FIG. 7A, the example pump device 700 can include a filter 740C disposed within the first fluid passageway 710. In another example, as shown in FIG. 7B, the example pump device 700 can include a filter 740C disposed within the second fluid passageway 720. In another example, as shown in FIG. 7C, the example pump device 700 can include a filter 740C disposed within the first fluid passageway 710 and another filter 740C disposed within the second fluid passageway 720.

Referring to FIG. 7A, the filter 740C can include an outer frame 750 that supports material within the frame that includes a plurality of small openings or passages through which fluid can pass but which have a threshold size that blocks particles having a characteristic size greater than the threshold size from passing through the filter 740C.

The outer frame 750 can be secured to the base plate 702 that defines the first fluid passageway 710. In some implementations, the base plate 702 can define a receptacle that receives the outer frame 750. In some implementations, the receptacle can have a lateral extent (e.g., a diameter) that is greater than the lateral extent of the first fluid passageway 710, such that when the outer frame 750 is disposed in the receptacle, an inner wall of the outer frame has a lateral extent that is similar to the lateral extent of the first fluid passageway 710. In some implementations, the outer frame can be press fit into the receptacle. In some implementations the outer frame 750 can be welded to the portion of the base plate 702 that defines the receptacle. In some implementations, after the outer frame 750 of the filter 740C is placed in the receptacle, a foil 742 can be placed over the outer frame 750 and then attached (e.g., welded) to the base plate 702.

In different implementations, the outer frame 750 can be made of different materials. For example, if the outer frame 750 is to be welded to a titanium base plate 702, the outer frame 750 can be made of titanium. In another example, if the outer frame 750 is to be securely press fit into a receptacle, the outer frame 750 can be made of a compliant material, for example, plastic, rubber, etc.

The material of the filter 740C supported by the outer frame 750, which includes a plurality of small openings or passages through fluid passes, can be made of different materials, which need not be identical or similar to the materials of the outer frame 750. For example, the material can include metal (e.g., titanium, gold, etc.). In another example the material can include ceramic material. In another example, the material can include plastic.

In some implementations, the thickness of the material of the filter, which includes the plurality of small openings or passages through which fluid passes, in the direction of the fluid flow through the filter can be greater than three times the mean lateral extent of the openings or passages through which the fluid passes. Thus, the openings or passages of the materials can operate more as tubes through which the fluid passes than as apertures in a thin plane of material. In some implementations, walls of the openings or passages of the material can be textured or treated to promote the adhesion of particulate matter, while also permitting the fluid to pass through the openings or passages. For example, the walls of the openings or passages can have a surface texture or roughness that facilitates the adhesion of particulate matter, and the service of the openings or passages can include a hydrophobic coating to encourage the passage of fluid through the openings or passages.

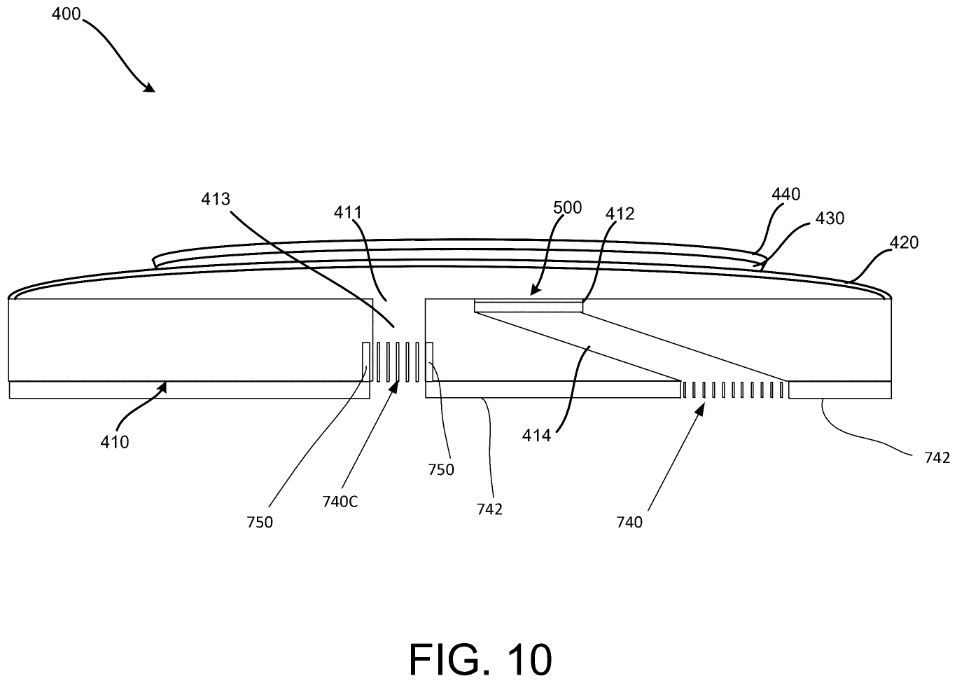

In addition to being used in the pumps described herein, the filters described herein also can be used in the valves described herein. For example, FIG. 10 is cross-sectional view of the valve device 400 shown in FIGS. 5A and 5B, but also including a filter 740 located at an end of the second fluid passageway 414 and a filter 740C located within the first fluid passageway 413. The filters described herein also may be utilized in other valve structures described herein.

It is desirable that the implantable fluid-operated inflatable device described herein can be implanted in a patient and used to provide safe, reliable, and successful therapeutic treatment to the patient for many years, for example, 10 or more years. It is also desirable that the device does not break or cause injury to the body of the patient in the case of misuse of the device by the patient or in the case of other unintended uses of the device. For example, in a device where the inflatable member is an elongate tubular member and is disposed within a penis or a neophallus of a person, if the person exerts too much pressure or the inflatable member becomes folded or otherwise compromised, it is desirable that the device release the pressure within the inflatable member before the inflatable member breaks or ruptures. Additionally, for example, in a device where the inflatable member is a loop or a cuff (such as in an artificial sphincter device), a physician may unknowingly insert a catheter and it would be desirable to release the pressure in the inflatable member before the inflatable member breaks or ruptures.

Accordingly, in some implementations, the piezoelectric elements that are used to operate the pumps and valves of the implantable fluid-operated inflatable devices 100, 200, disclosed herein may be used to release pressure in the inflatable member. For example, in some implementations, the piezoelectric elements may be used to move fluid from the inflatable member 104, 204 to the reservoir 102, 202 when it is detected that there has been a misuse or unintended use of the device. In some cases, the misuse or unintended use is detected by a pressure sensor (pressure sensor detects a high fluidic pressure in the system or in the inflatable member) or the misuse or unintended use may be detected by one of the piezoelectric elements (for example, when a voltage spike occurs on a piezoelectric element).

Referring back to FIG. 1, the electronic control system 108 drives the piezoelectric elements of the pumps and valves to move fluid within the device. Specifically, in some implementations, the driver circuitry 108E includes a piezoelectric driver that is configured to receive electrical energy from the power storage device 108D (a battery) and for generating a waveform of electrical energy that is provided to the piezoelectric elements of the pumps and valves to drive the piezoelectric elements to move the fluid within the system.

In some implementations, the power storage device 108D (the battery) can provide electrical energy at a maximum voltage of 5 V or less, for example, at a maximum of 4.4 V or less to the piezoelectric driver. The driver can step up the voltage and can output a waveform having a peak-to-peak voltage of greater than 50 V, for example, 100 V, to the piezoelectric element. In some implementations, the driver can include step up transformer circuitry configured for receiving a first voltage signal from the battery and for outputting a second voltage signal to the piezoelectric element, where the second voltage is greater than the first voltage.

When the piezoelectric element is associated with a pump of the implantable inflatable device 100, 200, the driver can output a periodic waveform that is used to repeatedly change a volume of a fluid chamber to cause fluid to be pumped through the fluid chamber from one location to another, for example, from a reservoir to an inflatable member or from the inflatable member to the reservoir. In some implementations, a frequency of the periodic waveform can be between 30 Hz and 60 Hz, for example, 40-50 Hz. In some implementations, the periodic waveform can be a sine wave. In some implementations, the periodic waveform can include a series of square pulses. In some implementations, the periodic waveform can include a repeated series of waves provided to the piezoelectric element, where the waves have a voltage that varies over time according to a function V=V(t) and where, unlike a sine wave, the second derivative of V divided by V (i.e., V″(t)/V(t)) is not equal to one but where, unlike a square wave, V(t) does not include discontinuities, at which the first derivative of V(t) approaches infinity. When comparing two waveforms having an identical frequency and an identical peak-to-peak amplitude, a first waveform in the form of a sine wave may be more energy-efficient, in terms of preserving energy in the battery, for driving the piezoelectric element than a second waveform in the form of a series of square pulses. More generally, a first waveform V₁(t) may be more energy-efficient, in terms of draining energy from the battery, for driving the piezoelectric element to pump a certain volume of fluid than a second waveform V₂(t) when the maximum of V″₁(t)/ V₁(t) is less than the maximum of V″₂(t)/ V₂(t).

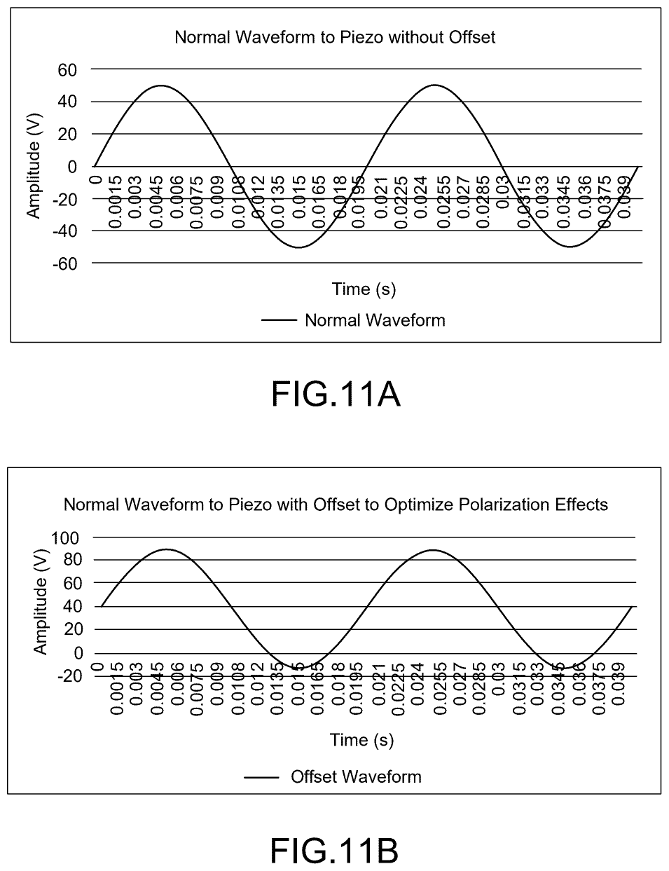

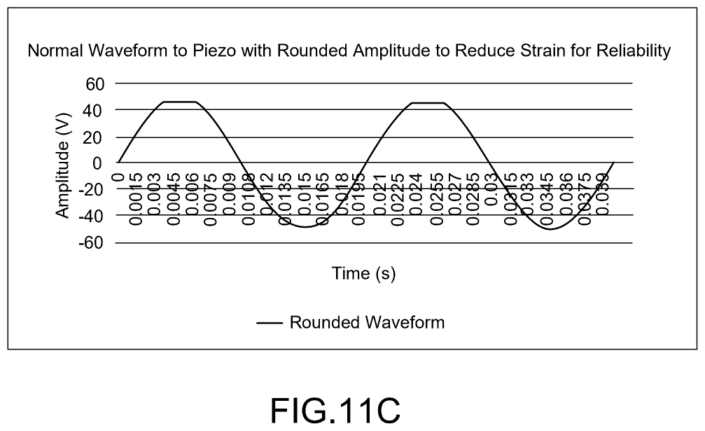

FIG. 11A is a graph of the voltage amplitude of an example waveform that can be a provided by the driver to the piezoelectric element to drive the piezoelectric element to cause a pump associated with the piezoelectric element to pump fluid. The waveform has an amplitude that varies over time according to a function V(t) that is approximated by a sine wave. In the example waveform of FIG. 11A, the voltage varies from −50 V to +50 V and has a frequency of 50 Hz.

FIG. 11B is a graph of the voltage amplitude of another example waveform that can be a provided by the driver to the piezoelectric element to drive the piezoelectric element to cause a pump associated with the piezoelectric element to pump fluid. The waveform has an amplitude that varies over time according to a function V(t) that is approximated by a sine wave having a frequency of 50 Hz. In contrast to the example waveform of FIG. 11A, in the example waveform of FIG. 11B, the average voltage over time is offset from zero, and the voltage varies from −12 V to +88 V. By offsetting the average voltage from zero, a polarization can be induced in the piezoelectric material, which can enhance the mechanical response of the piezoelectric material to the varying voltage of the waveform.

FIG. 11C is a graph of the voltage amplitude of another example waveform that can be a provided by the driver to the piezoelectric element to drive the piezoelectric element to cause a pump associated with the piezoelectric element to pump fluid. The waveform has an amplitude that varies over time according to a function V(t), has a frequency of 50 Hz, and a voltage that varies from −45 V to +45 V. In contrast to the example waveform of FIG. 11A, the example waveform of FIG. 11C is not approximated by sine wave but rather is approximated by a sine wave having a time-averaged value of zero, with the peak-to-peak amplitude of 100 V, except that for the times at which the amplitude would be greater than +45 V the amplitude is held fixed at a plateau of +45 V and except that for the times at which the amplitude would be less than −45 V the amplitude is held fixed at a plateau of −45 V. By including the +45 V and −45 V plateaus in the waveform, the waveform of FIG. 11C may be able to pump a substantially similar, or even a greater, amount of fluid as the waveform of FIG. 11A, while causing less mechanical strain on the material of the piezoelectric element, which may increase the reliability and longevity of the piezoelectric element. Because the fluid that is pumped by the piezoelectric-operated pump has a nonzero viscosity, the slightly smaller range of motion induced in the piezoelectric element by the application of the waveform of FIG. 11C, as compared to the application of the waveform of FIG. 11A, may result in a negligible difference in the amount of fluid pumped per cycle when the waveform of FIG. 11C is used instead of the waveform of FIG. 11A. Therefore, including short, fixed-voltage plateaus at the extrema of the voltage values of the waveform may increase the reliability and longevity of the piezoelectric element, while maintaining the pumping efficiency of the piezoelectric-operated pump.

The implantable device 100 also includes sensing devices 108F and as illustrated in FIG. 3 in implantable device 200 the sensing devices include one or more pressure sensors 212, 214, and 216 that can measure a pressure of fluid at one or more locations of the system. For example, a first pressure sensor 212 can be connected to a fluidic circuit between a piezoelectric pump and an inflatable member, where the pump supplies fluid from a reservoir and the inflatable member, to measure a fluid pressure in the inflatable member. A second pressure 214 sensor can be connected to a fluidic circuit between the piezoelectric pump and a valve, where the pump supplies fluid from a reservoir and the inflatable member and the valve is between the pump and the inflatable member, and configured to measure a fluid pressure in the fluidic circuit between the pump and the valve. A third pressure sensor 216 can be connected to a fluidic circuit between a reservoir and the piezoelectric pump to measure a fluid pressure in the reservoir.

The processor 108A can receive signals indicating the pressures of the various locations within the fluidic circuit. For example, the processor 108A can receive an indication of the pressure of the inflatable member from the first pressure sensor 212. In some implementations, the processor 108A receives signals indicating the pressure of the inflatable member while the inflatable member is in its inflated configuration from the first pressure sensor 212. An increase in the pressure within the inflatable member may indicate a misuse of the inflatable member or the implantable device by the patient or other unintended use of the implantable device.

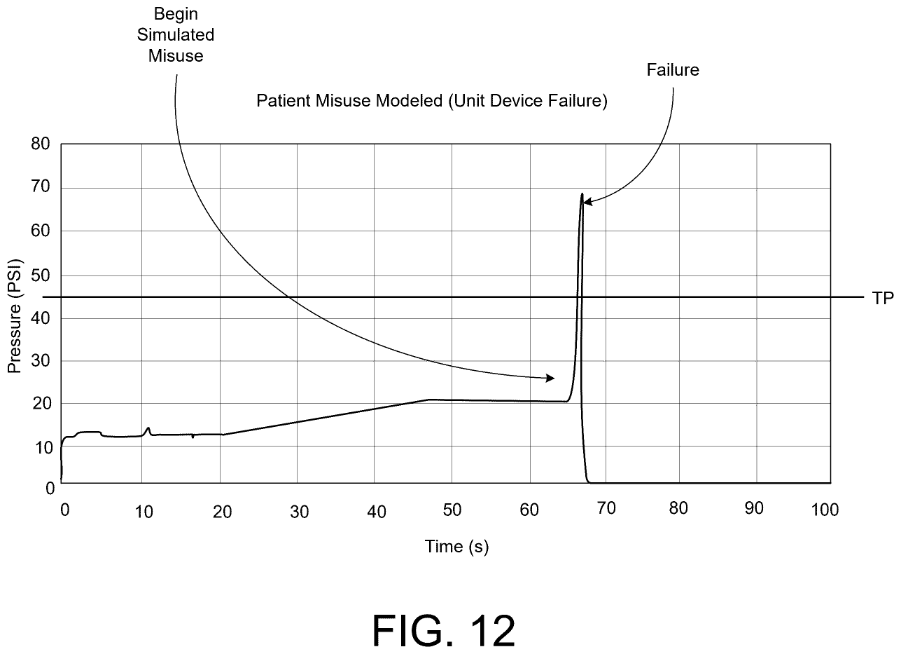

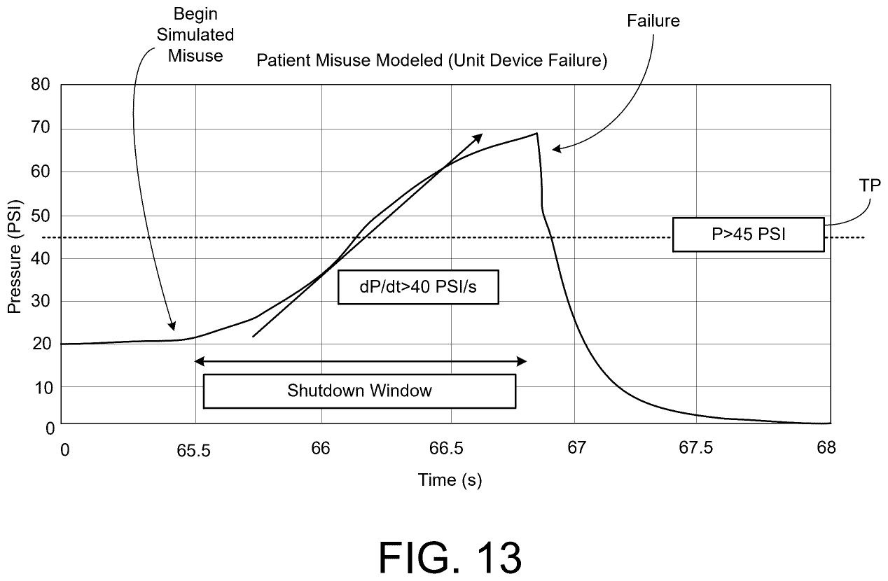

FIG. 12 is a graph that illustrates the pressure within the inflatable member. FIG. 12 is an expanded portion of the graph of FIG. 13. As illustrated in FIGS. 11 and 12, a sharp increase in the pressure within the inflatable member which occurs between 60 and 70 seconds may be indicative of patient misuse of the device or another unintended use of the device. For example, the pressure within the inflatable member may increase in such a manner when a patient attempts to bend or fold the inflatable member.

In some implementations, to help avoid damage to the implantable device (including the inflatable member), the processor 108A is configured to detect when the pressure within the inflatable member has reached a threshold pressure and cause the pressure to decrease by allowing or causing fluid to flow from the inflatable member to the reservoir. For example, in some implementations, when a threshold pressure (such as TP) is reached within the inflatable member, the processor 108A is configured to cause the driver circuitry to activate a piezoelectric pump (such as pump P2) to move or pump fluid from the inflatable member to the reservoir. For example, in some implementations, the processor is configured to cause the driver circuitry to provide a waveform of electrical energy from the power storage device 108D (such as a battery) to the piezoelectric pump (such as pump P2) to cause the piezoelectric pump to pump fluid from the inflatable member to the fluid reservoir. In some implementations, the threshold pressure is about 45 PSI (pounds per square inch). In other implementations, the threshold pressure is less than 45 PSI. In yet other implementations, the threshold pressure is more than 45 PSI.

In some implementations, the processor 108A is configured to detect when a pressure within the inflatable member has increased rapidly. For example, in some implementations, the processor 108A is configured to detect or identify when the change in pressure is high with respect to time (dP/dT) as illustrated in FIG. 13. In such cases, the processor is configured to cause the pressure in the inflatable member to decrease when the processor detects that the rate of change of the pressure is high or above a threshold rate of change. For example, in some implementations, when a threshold rate of change is reached within the inflatable member, the processor 108A is configured to cause the driver circuitry to activate a piezoelectric pump (such as pump P2) to move or pump fluid from the inflatable member to the reservoir. In some implementations, the threshold rate of change is 40 PSI (pounds per square inch) per second. In other implementations, the threshold rate of change is higher or lower than 40 PSI (pounds per square inch) per second.

In some implementations, when a threshold pressure (such as TP) or threshold rate of change is reached within the inflatable member, the processor 108A is configured to cause a valve (such as valve V2) to open to allow fluid to flow from the inflatable member to the reservoir. Accordingly, in some implementations, the pressure within the inflatable member may be lowered or decreased in advance of the inflatable member becoming damaged or causing injuring to the user.

In some implementations, the processor 108A or other component of the device (such as the driver circuitry 108E) is configured to detect a voltage spike on the piezoelectric elements (such as one of the piezoelectric pumps or valves). For example, a voltage spike on the piezoelectric elements may be detected when the piezoelectric elements are not active or are not currently pumping fluid within the system. In some cases, a voltage spike on one or more of the piezoelectric elements may indicate an increase in pressure within the fluidic circuit. In some implementations, processor 108A is configured to cause the pressure within the inflatable member to decrease by allowing or causing fluid to flow from the inflatable member to the reservoir when a voltage spike on one or more of the piezoelectric elements is detected.

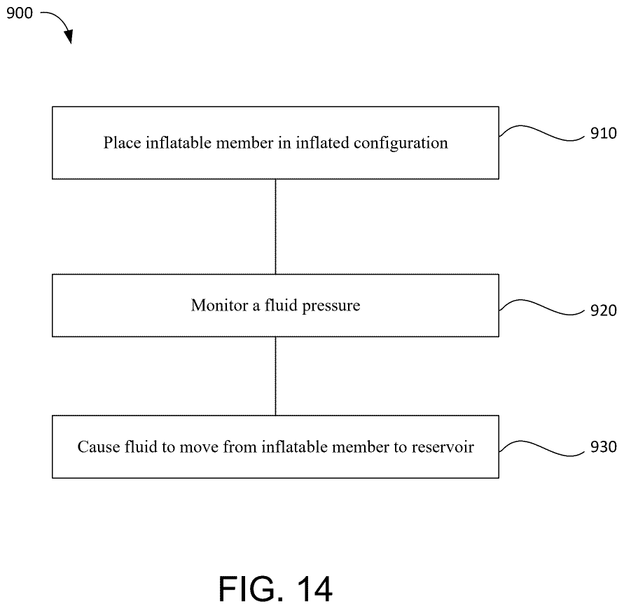

FIG. 14 is a flowchart of an example process 900 of operating an implantable fluid-operated device that includes a battery, a fluid reservoir, and an inflatable member. The process includes providing fluid to the inflatable member to place the inflatable member in an inflated configuration (910). The process also includes monitoring a fluid pressure in a fluidic circuit that includes the fluid reservoir, the piezoelectric pump, and the inflatable member after the inflatable member has been placed in the inflated configuration (920). The process also includes, based on a fluid pressure, causing fluid to move from the inflatable member to the fluid reservoir (930).

In some implementations, the implantable fluid-operated device includes a pressure sensor that may be used to monitor a fluid pressure in the fluidic circuit. In some implementations, the monitoring the fluid pressure may include detecting a voltage spike on a piezoelectric element of the implantable fluid-operated device. In some implementations, the piezoelectric element is a pump and in other implementations, the piezoelectric element is a valve.

In some implementations, the fluid is moved from the inflatable member to the fluid reservoir by pumping fluid with a pump (such as a piezoelectric pump) from the inflatable member to the reservoir.

While certain features of the described implementations have been illustrated as described herein, many modifications, substitutions, changes and equivalents will now occur to those skilled in the art. It is, therefore, to be understood that the will and in and in appended claims are intended to cover all such modifications and changes as fall within the scope of the embodiments.

What is claimed is:

1. An implantable device, comprising:
a battery configured for storing electrical energy;
a fluid reservoir;
an inflatable member configured to receive fluid to place the inflatable member in an inflated configuration;
a piezoelectric pump fluidically connected between the fluid reservoir and the inflatable member and configured to pump fluid from the inflatable member to the fluid reservoir;
driver circuitry configured for receiving electrical energy from the battery and for providing a waveform of electrical energy to drive the piezoelectric pump to pump fluid from the inflatable member to the fluid reservoir; and
a processor configured to, based on a fluid pressure detected within the inflatable member, cause the driver circuitry to provide a waveform of electrical energy from the battery to the piezoelectric pump to cause the piezoelectric pump to pump fluid from the inflatable member to the fluid reservoir.

2. The implantable device of claim 1, further comprising:
a pressure sensor configured to measure a fluid pressure in a fluidic circuit that includes the fluid reservoir, the piezoelectric pump, and the inflatable member.

3. The implantable device of claim 1, further comprising:
a pressure sensor fluidically connected between the piezoelectric pump and the inflatable member to measure a fluid pressure in the inflatable member.

4. The implantable device of claim 1, wherein the processor is further configured to determine when the fluid pressure detected within the inflatable member has exceeded a threshold value.

5. The implantable device of claim 1, wherein the processor is further configured to determine when a voltage spike occurs on the piezoelectric pump.

6. The implantable device of claim 1, further comprising:
a piezoelectric valve fluidically connected between the fluid reservoir and the inflatable member.

7. The implantable device of claim 1, further comprising:
a piezoelectric valve fluidically connected between the fluid reservoir and the inflatable member,
wherein the processor is further configured to determine when a voltage spike occurs on the piezoelectric valve.

8. An implantable device, comprising:
a battery configured for storing electrical energy;
a fluid reservoir;
an inflatable member configured to receive fluid to place the inflatable member in an inflated configuration;
a piezoelectric valve fluidically connected between the fluid reservoir and the inflatable member;
driver circuitry configured for receiving electrical energy from the battery and for providing a waveform of electrical energy to the piezoelectric valve; and
a processor configured to, based on a fluid pressure detected within the inflatable member, cause the driver circuitry to provide a waveform of electrical energy from the battery to the piezoelectric valve to cause the piezoelectric valve to allow fluid to pass from the inflatable member to the fluid reservoir.

9. The implantable device of claim 8, further comprising:
a pressure sensor configured to measure a fluid pressure in a fluidic circuit that includes the fluid reservoir, the piezoelectric valve, and the inflatable member.

10. The implantable device of claim 8, further comprising:
a pressure sensor fluidically connected between the piezoelectric valve and the inflatable member to measure a fluid pressure in the inflatable member.

11. The implantable device of claim 8, wherein the processor is further configured to determine when the fluid pressure detected within the inflatable member has exceeded a threshold value.

12. The implantable device of claim 8, wherein the processor is further configured to determine when a voltage spike occurs on the piezoelectric valve.

13. The implantable device of claim 8, further comprising:
a piezoelectric pump fluidically connected between the fluid reservoir and the inflatable member.

14. The implantable device of claim 8, further comprising:
a piezoelectric pump fluidically connected between the fluid reservoir and the inflatable member,
wherein the processor is further configured to determine when a voltage spike occurs on the piezoelectric pump.

15. The implantable device of claim 8, further comprising:
a first pressure sensor configured to measure a fluid pressure in a fluidic circuit that includes the fluid reservoir, the piezoelectric valve, and the inflatable member; and
a second pressure sensor configured to measure a fluid pressure in a fluidic circuit that includes the fluid reservoir, the piezoelectric valve, and the inflatable member.

16. A method of operating an implantable device that includes a battery, a fluid reservoir, an inflatable member, a piezoelectric pump fluidically connected between the fluid reservoir and the inflatable member, the method comprising:
providing fluid to the inflatable member to place the inflatable member in an inflated configuration;
monitoring a fluid pressure in a fluidic circuit that includes the fluid reservoir, the piezoelectric pump, and the inflatable member after the inflatable member has been placed in the inflated configuration; and
based on a fluid pressure, causing fluid to move from the inflatable member to the fluid reservoir.

17. The method of claim 16, wherein the implantable device includes a pressure sensor configured to measure a fluid pressure within the fluidic circuit, the monitoring includes monitoring the fluid pressure in the fluidic circuit via the pressure sensor.

18. The method of claim 16, wherein the monitoring the fluid pressure in the fluidic circuit includes detecting a voltage spike on the piezoelectric pump.

19. The method of claim 16, wherein the implantable device includes a piezoelectric valve, the monitoring the fluid pressure in the fluidic circuit includes detecting a voltage spike on the piezoelectric valve.

20. The method of claim 16, wherein the causing the fluid to move from the inflatable member to the fluid reservoir includes causing the piezoelectric pump to move the fluid from the inflatable member to the fluid reservoir.
